(19) United States
(12) Patent Application Publication
(10) Pub. No.: US 2025/0325708 A1
(43) Pub. Date: Oct. 23, 2025

(54) CRISPR-DCAS9-MEDIATED INDUCTION OF CD16/FCGR3A GENE EXPRESSION IN HUMAN NATURAL KILLER CELLS

(71) Applicants: Temple University-Of The Commonwealth System of Higher Education, Philadelphia, PA (US); Washington University in St. Louis, St. Louis, MO (US); Case Western Reserve University, Cleveland, OH (US)

(72) Inventors: Rafal Kaminski, Philadelphia, PA (US); Tricia H. Burdo, Dresher, PA (US); Liang Shan, Philadelphia, PA (US); Jonathan Karn, Philadelphia, PA (US)

(21) Appl. No.: 19/182,840

(22) Filed: Apr. 18, 2025

Related U.S. Application Data
(60) Provisional application No. 63/636,350, filed on Apr. 19, 2024.

(51) Int. Cl. A61K 48/00; C12N 9/22; C12N 15/11

(52) U.S. Cl. CPC A61K 48/0058; C12N 9/226; C12N 15/11; C12N 2310/20

(57) ABSTRACT

The present invention relates to systems and methods for increasing expression of CD16 in immune cells to improve their proliferation and cytolytic functions and method of use to treat or prevent diseases or disorders.

### CROSS REFERENCE TO RELATED APPLICATIONS

This application claims priority to U.S. Provisional Application No. 63/636,350, filed Apr. 19, 2024, which is hereby incorporated by reference herein in its entirety.

### STATEMENT REGARDING FEDERALLY SPONSORED RESEARCH OR DEVELOPMENT

This invention was made with government support under AI164568 awarded by the National Institutes of Health. The government has certain rights in the invention.

### REFERENCE TO A “SEQUENCE LISTING” SUBMITTED AS AN XML FILE

The present application hereby incorporates by reference the entire contents of the sequence listing xml document named “206017-0269-00US_Sequence_Listing.xml”. The xml file containing the Sequence Listing of the present application was created on Apr. 16, 2025 and is 11,818 bytes in size.

### BACKGROUND OF THE INVENTION

Fc-dependent antibody effector functions are essential to the antibody-mediated elimination of residual HIV-infected cells. In the past 10 years, a variety of broadly reactive neutralizing antibodies (bNAb) have been isolated from a small subset of people with HIV (PWH) and shown to act against a wide spectrum of viruses by targeting conserved regions on the HIV envelope trimer. Passive administration of the bNAbs reduced plasma HIV-1 RNA in untreated viremic patients, and delayed virus rebound in antiretroviral therapy (ART) treated patients after treatment interruption. In the RV144 HIV clinical vaccine trail using ALVAC-HIV and AIDSVAX gp120B/E, nNAbs targeting HIV-1 envelope gp120 VIV2 regions were associated with an estimated 31% protection against HIV acquisition, in the absence of neutralizing antibody responses and T cell responses.

In addition to Fab regions which determine antibody neutralizing activity, antibody Fc regions are indispensable to antibody effector functions, which trigger the killing of antibody-coated cells. To kill HIV-infected cells, the antibody Fab region binds to the envelope protein on the surface of infected cells, while its Fc region engages with Fc-gamma receptors (FcγR) on the innate immune effector cells. In nonhuman primate and humanized mouse models, Fc effector functions are required for bNAb-mediated clearance of infected cells, although it is not clear whether neutralizing activity also requires Fc functions. Fc-FcγR interaction can trigger the killing of infected cells through antibody-dependent cell-mediated cytotoxicity (ADCC) by natural killer (NK) cells and antibody-dependent cellular phagocytosis (ADCP) by phagocytes such as monocytes and macrophages. NK cells play a crucial role in ADCC, which involves the recognition and killing of virus-infected cells by antibodies bound to their surface. These antibodies can be recognized by the NK cell's CD16 receptor. NK cells provide a rapid response to HIV infection and contribute to antibody-mediated protection, but NK cell effector functions are impaired in active and chronic HIV infection, and only partially restored by ART.

Thus, there is a need in the art for improved compositions and methods for treating and preventing chronic infection. This invention satisfies this unmet need.

### SUMMARY OF THE INVENTION

In some embodiments, the present invention provides a genome editing system comprising a targeted transcriptional activator comprising a catalytically dead CRISPR Cas protein linked to a transcription activation domain, and at least one sgRNA molecule comprising nucleotide sequence that is complementary to the promoter of CD16.

In some embodiments, the at least one sgRNA molecule comprising nucleotide sequence that is complementary to the promoter of CD16 is selected from the group consisting of SEQ ID NO: 1-12.

In some embodiments, the at least one sgRNA molecule comprising nucleotide sequence that is complementary to the promoter of CD16 is selected from the group consisting of SEQ ID NO: 6, SEQ ID NO: 11, and SEQ ID NO: 12.

In some embodiments, wherein the system comprises an mRNA molecule encoding the catalytically dead CRISPR Cas protein linked to a transcription activation domain.

In some embodiments, the transcription activation domain comprises a fusion protein comprising VP64, p65 and Rta.

In some embodiments, the present invention provides methods of increasing the expression of CD16 in a subject, comprising administering to the subject: a) a genome editing system comprising a targeted transcriptional activator comprising a catalytically dead CRISPR Cas protein linked to a transcription activation domain; and b) at least one sgRNA molecule comprising nucleotide sequence that is complementary to the promoter of CD16.

In some embodiments, the at least one sgRNA molecule comprising nucleotide sequence that is complementary to the promoter of CD16 is selected from the group consisting of SEQ ID NO: 1-12.

In some embodiments, the system comprises an mRNA molecule encoding the catalytically dead CRISPR Cas protein linked to a transcription activation domain.

In some embodiments, the transcription activation domain comprises a fusion protein comprising VP64, p65 and Rta.

In some embodiments, the present invention provides methods of increasing the proliferation of immune cells in a subject, comprising administering to the subject: a) a genome editing system comprising a targeted transcriptional activator comprising a catalytically dead CRISPR Cas protein linked to a transcription activation domain; and b) at least one sgRNA molecule comprising nucleotide sequence that is complementary to the promoter of CD16.

In some embodiments, the at least one sgRNA molecule comprising nucleotide sequence that is complementary to the promoter of CD16 is selected from the group consisting of SEQ ID NO: 1-12.

In some embodiments, the system comprises an mRNA molecule encoding the catalytically dead CRISPR Cas protein linked to a transcription activation domain. In some embodiments, the transcription activation domain comprises a fusion protein comprising VP64, p65 and Rta.

In some embodiments, the immune cell is selected from the group consisting of a T cell, natural killer (NK) cell, myeloid cell, antigen presenting cell, dendritic cell, macrophage, and B cell.

In some embodiments, the immune cell is a NK cell.

In some embodiments, the present invention provides methods of treating or preventing a disease or disorder in a subject, comprising administering to the subject: a) a genome editing system comprising a targeted transcriptional activator comprising a catalytically dead CRISPR Cas protein linked to a transcription activation domain; and b) at least one sgRNA molecule comprising nucleotide sequence that is complementary to the promoter of CD16.

In some embodiments, the at least one sgRNA molecule comprising nucleotide sequence that is complementary to the promoter of CD16 is selected from the group consisting of SEQ ID NO: 1-12.

In some embodiments, the system comprises an mRNA molecule encoding the catalytically dead CRISPR Cas protein linked to a transcription activation domain.

In some embodiments, the transcription activation domain comprises a fusion protein comprising VP64, p65 and Rta.

In some embodiments, the disease or disorder is cancer or an infectious disease.

In some embodiments, the disease or disorder is HIV infection or AIDS.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts a representative schematic for achieving cure or post-treatment control by CRISPR.

FIG. 2 depicts a representative illustration of immune protection after HIV acquisition.

FIG. 3 depicts a representative schematic for the modulation of NK cell and T cell functions by CRISPR-Cas9.

FIG. 4 depicts a representative schematic for the repurposing CRISPR as an RNA-Guided Platform for sequence-specific control of gene expression.

FIG. 5 depicts a representative schematic demonstrating the reversal of NK exhaustion and increase of NK killing by modulation of CD16 by CRISPR, and that CD16 mediates antibody-dependent cell-mediated cytotoxicity (ADCC) of NK cells.

FIG. 6A and FIG. 6B depict a representative gRNA screen in K562-dCas9-VPR erythromyeloid cell line (CD16a) (FIG. 6A); qRT-PCR and immunolabeling/flow cytometry (FIG. 6B). FIG. 6A depicts a representative illustration of a RT-qPCR based CRISPR-Cas9 gRNA screen for human CD16 gene promoter. (top) Schematic of the promoter region of the human CD16 gene. The positions of CRISPR gRNAs binding sites in the promoter are shown as green arrows alternative transcription sites as yellow arrows, alternative promoters as blue arrows, and translation start site (ATG) as red arrow. (bottom) Results of RT-qPCR. K562-dCas9-VPR cells (stably expressing dCas9-VPR protein, purchased from Horizon) were electroporated with candidate CRISPR gRNAs. Three days later cells were harvested, RNA was extracted and used in RT-qPCRs using primers specific to human CD16 and GAPDH as a reference. FIG. 6A depicts representative qPCR results from electroporated K562 cells. Inconsistent results between trials necessitates repetition of the experiments. Guide 6 showed consistently high expression in multiple screens. Guides 11 and 12 were only screened once, but they showed high expression as well. FIG. 6B depicts a representative illustration of immunolabeling/flow cytometry analysis based CRISPR-Cas9 gRNA screen for human CD16 gene promoter. (top) Representative flow-cytometry images (Guava MiniCyte) showing surface expression of CD16 on control (CRISPR-NT) and CD16 gRNA12 treated K562-dCas9-VPR cells, anti-CD16-FITC antibody was used for immunolabeling, Y-axis Green channel, X-axis, Red channel. (bottom) Summary results of immunolabeling/flow-cytometry for cell surface CD16 expression on cells from the experiment shown in FIG. 6A. K562-dCas9-VPR cells (stably expressing dCas9-VPR protein, purchased from Horizon) were electroporated with candidate CRISPR gRNAs. Three days later cells were counted, immunolabelled with anti-CD16-FITC antibody (or Isotype-FITC for negative control), fixed, and analyzed on Guava MiniCyte flow cytometer for cell surface CD16-FITC positivity.

FIG. 7 depicts a representative summary of molecular targets for immune editing by CRISPR.

FIG. 8 depicts a representative illustration of antibody dependent cell cytotoxicity.

FIG. 9 depicts a representative workflow for analyzing cells via qRT-PCR and flow cytometry.

FIG. 10 depicts representative data demonstrating that immunophenotyping of K562 cells show no CD16 expression. These plots are representative to both pre-and post-electroporation with guide RNAs.

FIG. 11 depicts representative data demonstrating NK primary cell immunophenotyping post NK Isolation. Proportion of CD56+ cells mirrors what would be expected in whole blood.

FIG. 12 depicts a representative illustration of the FCG3Ra gene showing the 12 guide RNA's locations. Three promotors were considered when designing the guides: Pmed2, Pmed1 and Pprox. Guides 6, 11, and 12 (marked below) showed the most promising upregulation of the CD16 receptor.

FIG. 13A and FIG. 13B depict representative qPCR results from electroporated primary NK cells from human donors. Inconsistent results between trials necessitates repetition of the experiments. Donor 2 excluded due to technical issues. FIG. 14 depicts a representative schematic of human CD16 gene structure. Exons (coding sequence) are shown as white rectangles, transcription start sites (TSS), transcription termination sites (TTS), start codon (ATG), and stop codon (TGA) are shown in red font (top). The positions of CRISPR gRNA binding sites in the promoter are shown as green arrows, alternative transcription sites as yellow arrows, alternative promoters as blue arrows, and translation start site (ATG) as red arrow (bottom).

FIG. 15A and FIG. 15B depict representative data demonstrating the validation of lead CRISPR gRNAs targeting human CD16 gene promoter in primary human Natural Killer cells. (FIG. 15A) Results of RT-qPCR. Primary human NK cells were isolated from the blood of three different healthy donors and electroporated with dCas9-VPR mRNA together with the mix of lead CRISPR gRNAs targeting human CD16 gene promoter (gRNA 6, 11, and 12). Three days later cells were harvested, RNA was extracted and used in RT-qPCRs using primers specific to human CD16 and GAPDH as a reference. (FIG. 15B) Results of immunolabelling/flow-cytometry analysis of surface CD16 expression on the same cells. The % positivity and mean fluorescence intensity (MFI) of anti-CD16-PE immunolabeling is shown.

FIG. 16A through FIG. 16C depict representative data demonstrating the verification of dCas9-VPR/gRNA-mediated induction of CD16 expression in primary human NK cells by immunolabeling/flow cytometry. Primary human NK cells were isolated from the blood of a healthy donor using the EasySep CD56 positive selection kit (STEMCELL Technologies) and expanded for one week in ImmunoCult medium. Next, cells were electroporated with mRNA for dCas9-VPR (Horizon Biosciences) and non-targeting gRNAs (NT) or CD16/FCGR3A promoter targeting gRNAs. To verify the successful induction of CD16 expression, 24 h later, cells were stained with viability dye Zombie-NIR and immunolabeled with control IgG-PE and IgG-APC antibodies or anti-CD16-PE+anti-CD56-APC specific antibodies and analyzed for surface marker expression using a Guava MiniCyte 8 flow cytometer. The flow cytometry bivariate pseudocolor density plots for donor #6944 (FIG. 16A), donor #1377 (FIG. 16B), and donor #6949 (FIG. 16C) are shown. For the donors #1377 and #6949 day 2 and day 3 samples were collected as well.

FIG. 17A through FIG. 17C depict representative data demonstrating the verification of dCas9-VPR/gRNA-mediated induction of CD16 expression in primary human NK cells by immunolabeling/flow cytometry and RT-qPCR. Bar plots summarizing CD16 expression data from all three primary NK cell donors shown in FIG. 16. Surface CD16 expression was checked by immunolabeling/flow cytometry and was analyzed for CD16-labeling positivity (% of CD16-positive cells, FIG. 17A) and intensity (mean fluorescence intensity for CD16, FIG. 17B). Additionally, RNA was prepared from the same cells and analyzed by reverse transcription-qPCR for CD16 mRNA expression. GADPH mRNA was used as a housekeeping gene reference (FIG. 17C) Unpaired t-test, **p=0.0082

FIG. 18A through FIG. 18C depict representative data demonstrating a time course of dCas9-VPR/gRNA-mediated induction of CD16 expression in primary human NK cells by immunolabeling/flow cytometry and RT-qPCR. Bar plots summarizing CD16 expression data from two primary NK cell donors shown in FIG. 1 for which day 2 and day 3 samples were collected (donors #1377 and #6949). Surface CD16 expression was checked by immunolabeling/flow cytometry and was analyzed for CD16-labeling positivity (% of CD16-positive cells, FIG. 18A) and intensity (mean fluorescence intensity for CD16, FIG. 18B). Additionally, RNA was prepared from the same cells and analyzed by reverse transcription-qPCR for CD16 mRNA expression. GADPH mRNA was used as a housekeeping gene reference (FIG. 18C) One-way Anova (p=0.0073), Šídák's multiple comparisons test: *p=0316, **p=0.0089

### DETAILED DESCRIPTION

In one aspect, the invention relates to a genome editing system comprising mRNA for dCas9-VPR and at least one gRNA molecule comprising a targeting domain that is complementary with a target sequence of an CD16 gene.

In some embodiments, the at least one sgRNA molecule comprising nucleotide sequence that is complementary to the promoter of CD16 is selected from the group consisting of SEQ ID NO: 1-12. In some embodiments, the at least one sgRNA molecule comprising nucleotide sequence that is complementary to the promoter of CD16 is selected from the group consisting of SEQ ID NO:6, SEQ ID NO:11, and SEQ ID NO: 12.

In some embodiments, the present invention relates to a method of increasing the expression of a CD16 gene in a cell, comprising administering to the cell a genome editing system comprising mRNA for dCas9-VPR and at least one gRNA molecule comprising a second targeting domain that is complementary with a target sequence of an CD16 gene.

In some embodiments, the present invention relates to a method of treating or preventing HIV infection or AIDS in a subject, comprising administering to the subject a genome editing system comprising mRNA for dCas9-VPR and at least one gRNA molecule comprising a second targeting domain that is complementary with a target sequence of an CD16 gene.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “activate,” as used herein, means to induce or increase an activity or function, for example, about ten percent relative to a control value. Preferably, the activity is induced or increased by 50% compared to a control value, more preferably by 75%, and even more preferably by 95%. “Activate,” as used herein, also means to increase a molecule, a reaction, an interaction, a gene, an mRNA, and/or a protein's expression, stability, function or activity by a measurable amount or to increase entirely. Activators are compounds that, e.g., bind to, partially or totally induce stimulation, increase, promote, induce activation, activate, sensitize, or up regulate a protein, a gene, and an mRNA stability, expression, function and activity, e.g., agonists.

“Antisense” refers particularly to the nucleic acid sequence of the non-coding strand of a double stranded DNA molecule, or to a sequence which is substantially homologous to the non-coding strand. As defined herein, an antisense sequence is complementary to the sequence of a double stranded DNA molecule. It is not necessary that the antisense sequence be complementary solely to the coding portion of the coding strand of the DNA molecule. The antisense sequence may be complementary to regulatory sequences specified on the coding strand of a DNA molecule, which regulatory sequences control expression of the coding sequences.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of a compound which is sufficient to provide a beneficial effect to the subject to which the compound is administered. An “effective amount” of a delivery vehicle is that amount sufficient to effectively bind or deliver a compound.

The term “inhibit,” as used herein, means to suppress or block an activity or function, for example, about ten percent relative to a control value. Preferably, the activity is suppressed or blocked by 50% compared to a control value, more preferably by 75%, and even more preferably by 95%. “Inhibit,” as used herein, also means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

The terms “patient,” “subject,” “individual,” and the like are used interchangeably herein, and refer to any animal, or cells thereof whether in vitro or in vivo, amenable to the methods described herein. In certain non-limiting embodiments, the patient, subject or individual is a human.

A “therapeutic” treatment is a treatment administered to a subject who exhibits signs or symptoms of a disease or disorder, for the purpose of diminishing or eliminating those signs or symptoms.

As used herein, “treating a disease or disorder” means reducing the severity and/or frequency with which a sign or symptom of the disease or disorder is experienced by a patient.

The phrase “biological sample” as used herein, is intended to include any sample comprising a cell, a tissue, or a bodily fluid in which expression of a nucleic acid or polypeptide is present or can be detected. Samples that are liquid in nature are referred to herein as “bodily fluids.” Biological samples may be obtained from a patient by a variety of techniques including, for example, by scraping or swabbing an area of the subject or by using a needle to obtain bodily fluids. Methods for collecting various body samples are well known in the art.

As used herein, an “immunoassay” refers to any binding assay that uses an antibody capable of binding specifically to a target molecule to detect and quantify the target molecule.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

A “coding region” of a gene consists of the nucleotide residues of the coding strand of the gene and the nucleotides of the non-coding strand of the gene.

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Isolated” means altered or removed from the natural state. For example, a nucleic acid or a peptide naturally present in its normal context in a living animal is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “conjugated” refers to covalent attachment of one molecule to a second molecule.

“Variant” as the term is used herein, is a nucleic acid sequence or an amino acid sequence that differs in sequence from a reference nucleic acid sequence or amino acid sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis. In some embodiments, the variant sequence is at least about 85%, 86%, 87%, 88%, 89%, 90%, 91%, 92%, 93%, 94%, 95%, 96%, 97%, 98%, or 99%, identical to the reference sequence.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention relates generally to compositions and methods for increasing the expression or activity of CD16 on natural killer cells. In some embodiments, the invention provides a nucleic acid molecule encoding CD16 or an activator of CD16. In some embodiments, the methods enhance an immune response against an infectious disease (e.g., a bacterial or viral infection.) In some embodiments, the invention provides compositions and methods for the treatment or prevention of HIV infection or AIDS.

### Targeted Transcriptional Activator

In some embodiments, the invention relates to a composition comprising a targeted transcriptional activator for targeted activation of CD16. In one embodiment, the targeted transcriptional activator comprises targeting domain linked to a transcriptional activation domain. Exemplary transcription activation domains include, but are not limited to, VP16, VP64, VPR (VP16 or VP64, p65 and Rta), TET, SunTag, Synergistic

Activation Mediator (SAM), CREB binding protein (CBP), and SphI postoctamer homology (SPH). In some embodiments, the transcriptional activation domain comprises VP16, p65 and Rta. In some embodiments, the transcriptional activation domain comprises VP64, p65 and Rta.

In some embodiments, the targeting domain targets the transcriptional activator to a specific genomic location. In some embodiments, the targeting domain comprises a catalytically dead CRISPR Cas protein. Non-limiting examples of Cas proteins include Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2. Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, SpCas9, StCas9, NmCas9, SaCas9, CjCas9, CjCas9, AsCpf1, LbCpf1, FnCpf1, VRER SpCas9, VQR SpCas9, xCas9 3.7, homologs thereof, orthologs thereof, or modified versions thereof. In one embodiment, Cas protein is catalytically deficient (dCas).

In some embodiments, the Cas protein has DNA binding activity. In one embodiment, Cas protein is dCas9.

In some embodiments, the transcriptional activator comprises a dCas9-VP64-p65-Rta fusion. In some embodiments, the transcriptional activator comprises a dCas9.

CRISPR Single-Guide RNAs (sgRNAs)

In one aspect, the invention provides targeting nucleic acids, including a CRISPR single-guide RNA (sgRNA) for targeting the transcriptional activator to a target nucleic acid sequence. In some embodiments, the targeting nucleic acid is a targeting RNA oligo. In some embodiments, the RNA oligo comprises a guide sequence comprising a sequence having sufficient complementarity to the target sequence. In one embodiment, the targeting RNA oligo can be delivered in combination with the transcriptional activator to direct the transcriptional activator to a specific target genomic locus.

In some embodiments, the guide RNA is from 15 to 35 nt. In one embodiment, the guide RNA is at least 15 nucleotides. In one embodiment the guide RNA is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably the guide sequence is 10-30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

In some embodiments of CRISPR-Cas systems, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and advantageously tracr RNA is 30 or 50 nucleotides in length. However, an aspect of the invention is to reduce off-target interactions, e.g., reduce the guide interacting with a target sequence having low complementarity. Indeed, in the examples, it is shown that the invention involves mutations that result in the CRISPR-Cas system being able to distinguish between target and off-target sequences that have greater than 80% to about 95% complementarity, e.g., 83%-84% or 88-89% or 94-95% complementarity (for instance, distinguishing between a target having 18 nucleotides from an off-target of 18 nucleotides having 1, 2 or 3 mismatches). Accordingly, in the context of the present invention the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

CD16 sgRNA

In one aspect, the nucleic acid molecules of the disclosure comprise a targeted transcriptional activator and a sgRNA targeted to the promotor to drive the expression of CD16. In some embodiments, the targeted transcriptional activator is capable of driving expression of CD16 in an immune cell. Examples of immune cells that can be engineered for increased expression or secretion of CD16 include, but are not limited to, T cells (including killer T cells, helper T cells, regulatory T cells, and gamma delta T cells), natural killer (NK) cells, myeloid cells, antigen presenting cells, dendritic cells, macrophages, and B cells. In some embodiments, the immune cell is a natural killer (NK) cell. In some embodiments, the sgRNA is targeted to the promotor of human FCGR3A, also known as CD16, CD16-II, CD16A, FCG3, FCGR3, FCGRIII, FCR-10, FCRIII, FCRIIIA, FcGRIIIA, IGFR3, IMD20 (chromosome 1). In some embodiments, the sgRNA is targeted to the gene coding sequence of the FCGR3A gene. In some embodiments, the sgRNA is targeted to the promoter region upstream of transcription start sites of the FCGR3A gene.

In some embodiments, the sgRNA targeted to the CD16 promotor comprises a sequence at least 70%, at least 71%, at least 72%, at least 73%, at least 74%, at least 75%, at least 76%, at least 77%, at least 78%, at least 79%, 80%, at least 81%, at least 82%, at least 83%, at least 84%, at least 85%, at least 86%, at least 87%, at least 88%, at least 89%, 90%, at least 91%, at least 92%, at least 93%, at least 94%, at least 95%, at least 96%, at least 97%, at least 98%, or at least 99% identical to one of SEQ ID NOs: 1-12.

TABLE 1
Candidate CRISPR gRNAs targeting human
CD16 gene promoter region.

| gRNA | | Target site sequence | |
|---|---|---|---|
| # | gRNA name | (20n + PAM) | SEQ ID NO |
| g1 | 69rev | GTCTGTCAGCCTAGGAGCCGGGG | SEQ ID NO: 1 |
| g2 | SSJP5276.AH | GGACCTGGGTAACACGAGGAAGG | SEQ ID NO: 2 |
| g3 | SSJP5276.AJ | TCCTGGGATATGTAATCCACAGG | SEQ ID NO: 3 |
| g4 | SSJP5276.AM | ATCCACCCAGCACCAAGAATGGG | SEQ ID NO: 4 |
| g5 | 702forw | GCTCAGCCACAGACCTTTGAGGG | SEQ ID NO: 5 |
| g6 | SSJP5276.AG | AGGGAGCCCCACCATAGAACAGG | SEQ ID NO: 6 |
| g7 | 896forw | TTCCTCCTGTCTAGTCGGTTTGG | SEQ ID NO: 7 |
| g8 | 1.AA | AGGGACCAAACCGACTAGACAGG | SEQ ID NO: 8 |
| g9 | SSJP5276.AL | GTGACTTGTCCACTCCAGTGTGG | SEQ ID NO: 9 |
| g10 | SSJP5276.AB | TACGAATGAGACTCCCACGAAGG | SEQ ID NO: 10 |
| g11 | TMVD3793.AE | AGATGTCCACGAATCCACTGGGG | SEQ ID NO: 11 |
| g12 | TMVD3793.AJ | TAACCAACGTGGGGTTACGGGGG | SEQ ID NO: 12 |

Lead gRNAs: g6 (SEQ ID NO: 6), g11 (SEQ ID NO: 11), g12 (SEQ ID NO: 12)

### Nucleic Acids

The isolated nucleic acid sequences of the disclosure can be obtained using any of the many recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The nucleic acid molecules of the present invention can be modified to improve stability in serum or in growth medium for cell cultures. Modifications can be added to enhance stability, functionality, and/or specificity and to minimize immunostimulatory properties of the nucleic acid molecule of the invention. For example, in order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect function of the molecule.

In one embodiment of the present invention the nucleic acid molecule may contain at least one modified nucleotide analogue. For example, the ends may be stabilized by incorporating modified nucleotide analogues.

Non-limiting examples of nucleotide analogues include sugar-and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides the phosphoester group connecting to adjacent ribonucleotides is replaced by a modified group, e.g., of phosphothioate group. In exemplary sugar-modified ribonucleotides, the 2′ OH-group is replaced by a group selected from H, OR, R, halo, SH, SR, NH2, NHR, NR2 or ON, wherein R is C1-C6 alkyl, alkenyl or alkynyl and halo is F, Cl, Br or I.

Other examples of modifications are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino) propyl uridine, 5-bromo uridine; adenosine and/or guanosines modified at the 8 position, e.g., 8-bromo guanosine; deaza nucleotides, e.g., 7-deaza-adenosine; O-and N-alkylated nucleotides, e.g., N6-methyl adenosine are suitable. It should be noted that the above modifications may be combined.

In some instances, the nucleic acid molecule comprises at least one of the following chemical modifications: 2′-H, 2′-O-methyl, or 2′-OH modification of one or more nucleotides. In certain embodiments, a nucleic acid molecule of the invention can have enhanced resistance to nucleases. For increased nuclease resistance, a nucleic acid molecule, can include, for example, 2′-modified ribose units and/or phosphorothioate linkages. For example, the 2′ hydroxyl group (OH) can be modified or replaced with a number of different “oxy” or “deoxy” substituents. For increased nuclease resistance the nucleic acid molecules of the invention can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to a target.

In one embodiment, the nucleic acid molecule includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O-N-methylacetamido (2′-O-NMA). In one embodiment, the nucleic acid molecule includes at least one 2′-O-methyl-modified nucleotide, and in some embodiments, all of the nucleotides of the nucleic acid molecule include a 2′-O-methyl modification.

Nucleic acid agents discussed herein include otherwise unmodified RNA and DNA as well as RNA and DNA that have been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, or as occur naturally in the human body. The art has referred to rare or unusual, but naturally occurring, RNAs as modified RNAs, see, e.g., Limbach et al. (Nucleic Acids Res., 1994, 22: 2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from that which occur in nature, or different from that which occurs in the human body. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to be presented in the correct spatial relationship such that hybridization is substantially similar to what is seen with a ribophosphate backbone, e.g., non-charged mimics of the ribophosphate backbone. Modifications of the nucleic acid of the invention may be present at one or more of, a phosphate group, a sugar group, backbone, N-terminus, C-terminus, or nucleobase.

The present invention also includes a vector in which the isolated nucleic acid of the present invention is inserted. The art is replete with suitable vectors that are useful in the present invention.

In brief summary, the expression of natural or synthetic nucleic acids encoding a transcriptional activator of the disclosure is typically achieved by operably linking a nucleic acid encoding the transcriptional activator of the disclosure or portions thereof to a promoter and incorporating the construct into an expression vector. The vectors to be used are suitable for replication and, optionally, integration in eukaryotic cells. Typical vectors contain transcription and translation terminators, initiation sequences, and promoters useful for regulation of the expression of the desired nucleic acid sequence.

The vectors of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

The isolated nucleic acid of the invention can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the vector may be provided to a cell in the form of a viral vector. Viral vector technology is well known in the art and is described, for example, in Sambrook et al. (2012, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome or lipid nanoparticle. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

### Nanoparticle Formulations

In one embodiment, the composition of the invention may comprise a nanoparticle, including but not limited to a lipid nanoparticle (LNP), comprising a transcriptional activator, a sgRNA, or a combination thereof, of the invention, or a LNP comprising a nucleic acid encoding a transcriptional activator, a sgRNA, or a combination thereof, of the invention. In some embodiments, the composition comprises an mRNA molecule that encodes all or part of a transcriptional activator of the invention. In one embodiment, the immunogenic composition of the invention may comprise a composition comprising a combination of NA antibodies of the invention, or a LNP comprising one or more nucleic acid molecules encoding a combination of NA antibodies of the invention. In one embodiment, the immunogenic composition of the invention may comprise a composition comprising a combination of LNP, wherein the combination of LNP comprises one or more nucleic acid molecules encoding a combination of NA antibodies of the invention.

In one embodiment, the LNP comprises or encapsulates an mRNA molecule encoding a transcriptional activator and at least one sgRNA of SEQ ID NO:1-12, or a fragment or variant thereof.

In one embodiment, the composition further comprises one or more additional immunostimulatory agents. Immunostimulatory agents include, but are not limited to, an additional antigen or antigen binding molecule, an immunomodulator, or an adjuvant.

### Excipients and Other Components of the Composition

The composition may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the composition. The composition may also include a transfection facilitating agent such as lipids, liposomes, including lecithin liposomes or other liposomes known in the art, as a DNA-liposome mixture (see for example W09324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The composition may further comprise a genetic facilitator agent as described in U.S. Ser. No. 021,579 filed Apr. 1, 1994, which is fully incorporated by reference.

The composition can be formulated according to the mode of administration to be used. An injectable pharmaceutical composition can be sterile, pyrogen free and particulate free. An isotonic formulation or solution can be used. Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polycations or polyanions.

### Treatment Methods

In some embodiments, the invention provides a method for treatment or prevention of a disease or disorder which would benefit from an increase in NK cell function or activity. Exemplary diseases and disorders that can be treated using the compositions and methods of the invention include, but are not limited to cancer and infectious diseases.

The following are non-limiting examples of cancers that can be diagnosed or treated by the disclosed methods and compositions: acute lymphoblastic leukemia, acute myeloid leukemia, adrenocortical carcinoma, appendix cancer, basal cell carcinoma, bile duct cancer, bladder cancer, bone cancer, brain and spinal cord tumors, brain stem glioma, brain tumor, breast cancer, bronchial tumors, burkitt lymphoma, carcinoid tumor, central nervous system atypical teratoid/rhabdoid tumor, central nervous system embryonal tumors, central nervous system lymphoma, cerebellar astrocytoma, cerebral astrocytoma/malignant glioma, cerebral astrocytotna/malignant glioma, cervical cancer, childhood visual pathway tumor, chordoma, chronic lymphocytic leukemia, chronic myelogenous leukemia, chronic myeloproliferative disorders, colon cancer, colorectal cancer, craniopharyngioma, cutaneous cancer, cutaneous t-cell lymphoma, endometrial cancer, ependymoblastoma, ependymoma, esophageal cancer, ewing family of tumors, extracranial cancer, extragonadal germ cell tumor, extrahepatic bile duct cancer, extrahepatic cancer, eye cancer, fungoides, gallbladder cancer, gastric (stomach) cancer, gastrointestinal cancer, gastrointestinal carcinoid tumor, gastrointestinal stromal tumor (gist), germ cell tumor, gestational cancer, gestational trophoblastic tumor, glioblastoma, glioma, hairy cell leukemia, head and neck cancer, hepatocellular (liver) cancer, histiocytosis, hodgkin lymphoma, hypopharyngeal cancer, hypothalamic and visual pathway glioma, hypothalamic tumor, intraocular (eye) cancer, intraocular melanoma, islet cell tumors, kaposi sarcoma, kidney (renal cell) cancer, langerhans cell cancer, langerhans cell histiocytosis, laryngeal cancer, leukemia, lip and oral cavity cancer, liver cancer, lung cancer, lymphoma, macroglobulinemia, malignant fibrous histiocvtoma of bone and osteosarcoma, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, mycosis, myelodysplastic syndromes, myelodysplastic/myeloproliferative diseases, myelogenous leukemia, myeloid leukemia, myeloma, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma, osteosarcoma and malignant fibrous histiocytoma of bone, ovarian, ovarian cancer, ovarian epithelial cancer, ovarian germ cell tumor, ovarian low malignant potential tumor, pancreatic cancer, papillomatosis, paraganglioma, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm, plasma cell neoplasm/multiple myeloma, pleuropulmonary blastoma, primary central nervous system cancer, primary central nervous system lymphoma, prostate cancer, rectal cancer, renal cell (kidney) cancer, renal pelvis and ureter cancer, respiratory tract carcinoma involving the nut gene on chromosome 15, retinoblastoma, rhabdomyosarcoma, salivary gland cancer, sarcoma, sezary syndrome, skin cancer (melanoma), skin cancer (nonmelanoma), skin carcinoma, small cell lung cancer, small intestine cancer, soft tissue cancer, soft tissue sarcoma, squamous cell carcinoma, squamous neck cancer, stomach (gastric) cancer, supratentorial primitive neuroectodermal tumors, supratentorial primitive neuroectodermal tumors and pineoblastoma, T-cell lymphoma, testicular cancer, throat cancer, thymoma and thymic carcinoma, thyroid cancer, transitional cell cancer, transitional cell cancer of the renal pelvis and ureter, trophoblastic tumor, urethral cancer, uterine cancer, uterine sarcoma, vaginal cancer, visual pathway and hypothalamic glioma, vulvar cancer, waldenstrom macroglobulinemia, and wilms tumor.

### Bacterial Infections

In one embodiment, the infectious disease or disorder is associated with a bacterium. In some embodiments, the bacterium can be from any one of the following phyla: Acidobacteria, Actinobacteria, Aquificae, Bacteroidetes, Caldiserica, Chlamydiae, Chlorobi, Chloroflexi, Chrysiogenetes, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospira, Planctomycetes, Proteobacteria, Spirochaetes, Synergistetes, Tenericutes, Thermodesulfobacteria, Thermotogae, and Verrucomicrobia.

The bacterium can be a gram-positive bacterium or a gram-negative bacterium. The bacterium can be an aerobic bacterium or an anerobic bacterium. The bacterium can be an autotrophic bacterium or a heterotrophic bacterium. The bacterium can be a mesophile, a neutrophile, an extremophile, an acidophile, an alkaliphile, a thermophile, a psychrophile, an halophile, or an osmophile.

The bacterium can be an anthrax bacterium, an antibiotic resistant bacterium, a disease-causing bacterium, a food poisoning bacterium, an infectious bacterium, Salmonella bacterium, Staphylococcus bacterium, Streptococcus bacterium, or tetanus bacterium. The bacterium can be a mycobacteria, Clostridium tetani, Yersinia pestis, Bacillus anthracis, methicillin-resistant Staphylococcus aureus (MRSA), or Clostridium difficile. The bacterium can be mycobacterium tuberculosis or listeria.

Viral Infections In one embodiment, the infectious disease or disorder is associated with a bacterium. In some embodiments, the virus is from one of the following families: Adenoviridae, Arenaviridae, Bunyaviridae, Caliciviridae, Coronaviridae, Filoviridae, Hepadnaviridae, Herpesviridae, Orthomyxoviridae, Papovaviridae, Paramyxoviridae, Parvoviridae, Picornaviridae, Poxviridae, Reoviridae, Retroviridae, Rhabdoviridae, or Togaviridae. The viral antigen can be from human immunodeficiency virus (HIV), Chikungunya virus (CHIKV), dengue fever virus, papilloma viruses, for example, human papilloma virus (HPV), polio virus, hepatitis viruses, for example, hepatitis A virus (HAV), hepatitis B virus (HBV), hepatitis C virus (HCV), hepatitis D virus (HDV), and hepatitis E virus (HEV), smallpox virus (Variola major and minor), vaccinia virus, influenza virus, rhinoviruses, equine encephalitis viruses, rubella virus, yellow fever virus, Norwalk virus, hepatitis A virus, human T-cell leukemia virus (HTLV-I), hairy cell leukemia virus (HTLV-II), California encephalitis virus, Hanta virus (hemorrhagic fever), rabies virus, Ebola fever virus, Marburg virus, measles virus, mumps virus, respiratory syncytial virus (RSV), herpes simplex 1 (oral herpes), herpes simplex 2 (genital herpes), herpes zoster (varicella-zoster, a.k.a., chickenpox), cytomegalovirus (CMV), for example human CMV, Epstein-Barr virus (EBV), flavivirus, foot and mouth disease virus, lassa virus, arenavirus, severe acute respiratory syndrome-related coronavirus (SARS), Middle East respiratory syndrome-related coronavirus (MERS), severe acute respiratory syndrome-related coronavirus 2 (SARS COV 2) or a cancer causing virus. In some embodiments, the viral antigen can be from HIV, HBV, HCV, HTLV-1, or influenza.

### Parasitic Infections

In one embodiment, the infectious disease or disorder is associated with a parasite. In some embodiments, the parasite can be a protozoa, helminth, or ectoparasite. The helminth (i.e., worm) can be a flatworm (e.g., flukes and tapeworms), a thorny-headed worm, or a round worm (e.g., pinworms). The ectoparasite can be lice, fleas, ticks, and mites.

The parasite can be any parasite causing any one of the following diseases: Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Malaria, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, and Trichuriasis.

The parasite can be Acanthamoeba, Anisakis, Ascaris lumbricoides, Botfly, Balantidium coli, Bedbug, Cestoda (tapeworm), Chiggers, Cochliomyia hominivorax, Entamoeba histolytica, Fasciola hepatica, Giardia lamblia, Hookworm, Leishmania, Linguatula serrata, Liver fluke, Loa loa, Paragonimus—lung fluke, Pinworm, Plasmodium falciparum, Schistosoma, Strongyloides stercoralis, Mite, Tapeworm, Toxoplasma gondii, Trypanosoma, Whipworm, or Wuchereria bancrofti. In some embodiments, the parasite can be any parasite cause any one of the following disease: malaria, toxoplasmosis, and leishmaniasis.

### Fungal Infection

In one embodiment, the infectious disease or disorder is associated with a fungus. In some embodiments, the fungus can be Aspergillus species, Blastomyces dermatitidis, Candida yeasts (e.g., Candida albicans), Coccidioides, Cryptococcus neoformans, Cryptococcus gattii, dermatophyte, Fusarium species, Histoplasma capsulatum, Mucoromycotina, Pneumocystis jirovecii, Sporothrix schenckii, Exserohilum, or Cladosporium.

In one aspect, the invention provides a method for preventing in a subject, a disease or disorder, by administering to the subject a composition described herein. Administration of a prophylactic agent can occur prior to the manifestation of symptoms characteristic of the disease or disorder, such that the disease or disorder is prevented or delayed in its progression.

In some embodiments, the method comprises administering an effective amount of a composition described herein to a subject diagnosed with, suspected of having, or at risk for developing cancer or an infectious disease or disorder. In one embodiment, the composition is administered systemically to the subject.

The composition of the invention may be administered to a patient or subject in need in a wide variety of ways. Modes of administration include intraoperatively intravenous, intravascular, intramuscular, subcutaneous, intracerebral, intraperitoneal, soft tissue injection, surgical placement, arthroscopic placement, and percutaneous insertion, e.g., direct injection, cannulation or catheterization. Any administration may be a single application of a composition of invention or multiple applications. Administrations may be to single site or to more than one site in the individual to be treated. Multiple administrations may occur essentially at the same time or separated in time.

Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions of the present invention may be administered in a manner appropriate to the disease to be treated (or prevented). The quantity and frequency of administration will be determined by such factors as the condition of the subject, and the type and severity of the subject's disease, although appropriate dosages may be determined by clinical trials.

When “therapeutic amount” is indicated, the precise amount of the compositions of the present invention to be administered can be determined by a physician with consideration of individual differences in age, weight, disease type, extent of disease, and condition of the patient (subject).

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In one embodiment, the compositions of the present invention are administered to a patient by intradermal or subcutaneous injection. In another embodiment, the compositions of the present invention are administered by i.v. injection.

The pharmaceutical compositions useful for practicing the invention may be administered to deliver a dose of from 1 ng/kg/day and 100 mg/kg/day. In one embodiment, the invention envisions administration of a dose which results in a concentration of the compound of the present invention from 1 μM and 10 μM in a mammal.

Typically, dosages which may be administered in a method of the invention to a mammal range in amount from 0.5 μg to about 50 mg per kilogram of body weight of the mammal, while the precise dosage administered will vary depending upon any number of factors, including but not limited to, the type of mammal and type of disease state being treated, the age of the mammal and the route of administration. In one embodiment, the dosage will vary from about 1 μg to about 50 mg per kilogram of body weight of the mammal. In one embodiment, the dosage will vary from about 1 mg to about 10 mg per kilogram of body weight of the mammal.

The compound may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

In one embodiment, the invention provides methods of treating or preventing cancer, or of treating and preventing, aiding in the prevention, and/or reducing the risk of an infectious disease in an individual.

One aspect of the invention provides a method of preventing, aiding in the prevention, and/or reducing the risk of HIV infection or a disease or disorder associated therewith (e.g., AIDS).

In some embodiments of treating or preventing an infectious disease in an individual in need thereof, a second agent is administered to the individual, such as an antibody, a CAR molecule, an antigen or an immunogen for promoting an immune response against the infectious disease.

Another aspect of the invention provides methods of inducing antibody-dependent cellular cytotoxicity (ADCC) in a subject.

### Pharmaceutical Compositions

The present invention also provides various pharmaceutical compositions comprising an activator of the present invention. In some embodiments, the composition of the invention activates CD16.

In various embodiments, the present invention includes CD16 activator compositions for the use in treating or preventing a disease or disorder. One non-limiting example of a disease or disorder where elevated levels or activity of CD16 is desired which can be treated or prevented with the compositions and methods of the invention includes infectious diseases. In various embodiments, the CD16 activator compositions of the invention increase the amount of CD16 polypeptide, the amount of CD16 protein, the amount of CD16 mRNA, the amount of CD16 activity, or a combination thereof.

It will be understood by one skilled in the art, based upon the disclosure provided herein, that an increase in the level of CD16 encompasses the increase in CD16 expression, including transcription, translation, or both, and also encompasses promoting the stabilization of CD16, including at the RNA level (e.g., RNAi, shRNA, etc.) and at the protein level (e.g., Ubiquitination, etc.) The skilled artisan will also appreciate, once armed with the teachings of the present invention, that an increase in the level of CD16 includes an increase in a CD16 activity (e.g., enzymatic activity, substrate binding activity, receptor binding activity, etc.). Thus, increasing the level or activity of CD16 includes, but is not limited to, increasing transcription, translation, or both, of a nucleic acid encoding CD16; and it also includes increasing any activity of a CD16 polypeptide, or peptide fragment thereof, as well.

One skilled in the art, based upon the disclosure provided herein, would understand that the invention is useful in treating or preventing a disease or disorder or antibody-dependent cellular cytotoxicity (ADCC) in a subject in need thereof. In various embodiments, the disease or disorder treatable or preventable using the compounds of the invention is a chronic infectious disease. In some embodiments, the disease or disorder is HIV, HBV, HCV, HTLV-1, or influenza.

In one aspect, the present invention provides compositions comprising mRNA for dCas9-VPR and at least one gRNA molecule comprising a targeting domain that is complementary with a target sequence of an CD16 gene. In some embodiments, the at least one sgRNA molecule comprising nucleotide sequence that is complementary to the promoter of CD16 is selected from the group consisting of SEQ ID NO: 1-12. In some embodiments, the at least one sgRNA molecule comprising nucleotide sequence that is complementary to the promoter of CD16 is selected from the group consisting of SEQ ID NO:6, SEQ ID NO:11, and SEQ ID NO:12.

In various aspects, the composition comprises: one or more activator of the present invention and one or more stabilizers. In various embodiments, the stabilizer to compound weight ratio is less than 50%. In one embodiment, the stabilizer comprises a biocompatible polymer. Examples of stabilizers include, but are not limited to, biocompatible polymer, a biodegradable polymer, a multifunctional linker, starch, modified starch, and starch derivatives, gums, including but not limited to polymers, polypeptides, albumin, amino acids, thiols, amines, carboxylic acid and combinations or derivatives thereof, citric acid, xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum, polycarbonates (linear polyesters of carbonic acid); microporous materials (bisphenol, a microporous poly (vinylchloride), micro-porous polyamides, microporous modacrylic copolymers, microporous styrene-acrylic and its copolymers); porous polysulfones, halogenated poly (vinylidene), polychloroethers, acetal polymers, polyesters prepared by esterification of a dicarboxylic acid or anhydride with an alkylene polyol, poly (alkylenesulfides), phenolics, polyesters, asymmetric porous polymers, cross-linked olefin polymers, hydrophilic microporous homopolymers, copolymers or interpolymers having a reduced bulk density, and other similar materials, poly (urethane), cross-linked chain-extended poly (urethane), poly (imides), poly (benzimidazoles), collodion, regenerated proteins, semi-solid cross-linked poly (vinylpyrrolidone), monomeric, dimeric, oligomeric or long-chain, copolymers, block polymers, block co-polymers, polymers, PEG, dextran, modified dextran, polyvinylalcohol, polyvinylpyrollidone, polyacrylates, polymethacrylates, polyanhydrides, polypeptides, albumin, alginates, amino acids, thiols, amines, carboxylic acids, or combinations thereof.

The compositions may be formulated in a pharmaceutically acceptable excipient, such as wetting agents, buffers, disintegrants, binders, fillers, flavoring agents and liquid carrier media such as sterile water, water/ethanol etc. The compositions should be suitable for administration either by topical administration or injection or inhalation or catheterization or instillation or transdermal introduction into any of the various body cavities including the alimentary canal, the vagina, the rectum, the bladder, the ureter, the urethra, the mouth, etc. For oral administration, the pH of the composition is preferably in the acid range (e.g., 2 to 7) and buffers or pH adjusting agents may be used. The contrast media may be formulated in conventional pharmaceutical administration forms, such as tablets, capsules, powders, solutions, dispersion, syrups, suppositories etc.

The compositions of the invention can be formulated and administered to a subject, as now described. The invention encompasses the preparation and use of pharmaceutical compositions comprising the compositions of the invention useful for the delivery of a therapeutic agent to a cell. The invention also encompasses the preparation and use of pharmaceutical compositions comprising the compositions of the invention useful for the treatment of a disease or disorder. The invention also encompasses the preparation and use of pharmaceutical compositions comprising the compositions of the invention useful for improved cell penetration.

Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

In various embodiments, the pharmaceutical compositions useful in the methods of the invention may be administered, by way of example, systemically, parenterally, or topically, such as, in oral formulations, inhaled formulations, including solid or aerosol, and by topical or other similar formulations. In addition to the appropriate therapeutic composition, such pharmaceutical compositions may contain pharmaceutically acceptable carriers and other ingredients known to enhance and facilitate drug administration. Other possible formulations, such as nanoparticles, liposomes, resealed erythrocytes, and immunologically based systems may also be used to administer an appropriate modulator thereof, according to the methods of the invention.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single-or multi-dose unit.

Pharmaceutical compositions that are useful in the methods of the invention may be prepared, packaged, or sold in formulations suitable for oral, rectal, vaginal, parenteral, topical, pulmonary, intranasal, buccal, intravenous, ophthalmic, intrathecal and other known routes of administration. Other contemplated formulations include projected nanoparticles, liposomal preparations, resealed erythrocytes containing the active ingredient (e.g., a CD16 activator), and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient (e.g., the CD16 activator), the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient. In various embodiments, the composition comprises at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25%, at least about 26%, at least about 27%, at least about 28%, at least about 29%, at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% (w/w) active ingredient.

In addition to the active ingredient, a pharmaceutical composition of the invention may further comprise one or more additional pharmaceutically active agents.

Controlled-or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

A tablet comprising the active ingredient (e.g., a CD16 activator) may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycolate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent.

Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

Parenteral administration of a pharmaceutical composition includes any route of administration characterized by physical breaching of a tissue of an individual and administration of the pharmaceutical composition through the breach in the tissue. Parental administration can be local, regional or systemic. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intravenous, intraocular, intravitreal, subcutaneous, intraperitoneal, intramuscular, intradermal, intrasternal injection, and intratumoral. Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono-or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container.

Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. In some embodiments, dry powder compositions include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (in some embodiments having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers.

Such a formulation is administered in the manner in which snuff is taken i.e. by rapid inhalation through the nasal passage from a container of the powder held close to the nares. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 nanomaters to about 2000 micrometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa.

Administration of the compounds of the present invention or the compositions thereof may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems which are well known to the art.

One or more suitable unit dosage forms having the therapeutic agent(s) of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including parenteral, including by intravenous and intramuscular routes, as well as by direct injection into the diseased tissue. For example, the therapeutic agent may be directly injected into the muscle. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans. Such formulations include the use of adjuvants such as muramyl dipeptide derivatives (MDP) or analogs that are described in U.S. Pat. Nos. 4,082,735; 4,082,736; 4,101,536; 4,185,089; 4,235,771; and 4,406,890. Other adjuvants, which are useful, include alum (Pierce Chemical Co.), lipid A, trehalose dimycolate and dimethyldioctadecylammonium bromide (DDA), Freund's adjuvant, and IL 12. Other components may include a polyoxypropylene-polyoxyethylene block polymer (Pluronic®), a non-ionic surfactant, and a metabolizable oil such as squalene (U.S. Patent No. 4,606,918).

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the composition of the present invention may be delivered via various routes and to various sites in a mammal body to achieve a particular effect (see, e.g., Rosenfeld et al., 1991; Rosenfeld et al., 1991a; Jaffe et al., supra; Berkner, supra). One skilled in the art will recognize that although more than one route can be used for administration, a particular route can provide a more immediate and more effective reaction than another route. In one embodiment, the composition described above is administered to the subject by subretinal injection. In other embodiments, the composition is administered by intravitreal injection. Other forms of administration that may be useful in the methods described herein include, but are not limited to, direct delivery to a desired organ (e.g., the eye), oral, inhalation, intranasal, intratracheal, intravenous, intramuscular, subcutaneous, intradermal, and other parental routes of administration. Additionally, routes of administration may be combined, if desired. In another embodiments, route of administration is subretinal injection or intravitreal injection.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the composition in the particular host.

The pharmaceutical compositions useful for practicing the invention may be administered to deliver a dose of at least about 1 ng/kg, at least about 5 ng/kg, at least about 10 ng/kg, at least about 25 ng/kg, at least about 50 ng/kg, at least about 100 ng/kg, at least about 500 ng/kg, at least about 1 μg/kg, at least about 5 μg/kg, at least about 10 μg/kg, at least about 25 μg/kg, at least about 50 μg/kg, at least about 100 μg/kg, at least about 500 μg/kg, at least about 1 mg/kg, at least about 5 mg/kg, at least about 10 mg/kg, at least about 25 mg/kg, at least about 50 mg/kg, at least about 100 mg/kg, at least about 200 mg/kg, at least about 300 mg/kg, at least about 400 mg/kg, and at least about 500 mg/kg of body weight of the subject.

In some embodiments, the pharmaceutical compositions useful for practicing the invention may be administered to deliver a dose of no more than about 1 ng/kg, no more than about 5 ng/kg, no more than about 10 ng/kg, no more than about 25 ng/kg, no more than about 50 ng/kg, no more than about 100 ng/kg, no more than about 500 ng/kg, no more than about 1 μg/kg, no more than about 5 μg/kg, no more than about 10 μg/kg, no more than about 25 μg/kg, no more than about 50 μg/kg, no more than about 100 μg/kg, no more than about 500 μg/kg, no more than about 1 mg/kg, no more than about 5 mg/kg, no more than about 10 mg/kg, no more than about 25 mg/kg, no more than about 50 mg/kg, no more than about 100 mg/kg, no more than about 200 mg/kg, no more than about 300 mg/kg, no more than about 400 mg/kg, and no more than about 500 mg/kg of body weight of the subject. Also contemplated are dosage ranges between any of the doses disclosed herein.

Typically, dosages which may be administered in a method of the invention to a subject, in some embodiments a human, range in amount from 0.5 μg to about 100 g per kilogram of body weight of the subject. While the precise dosage administered will vary depending upon any number of factors, including but not limited to, the type of subject and type of disease state being treated, the age of the subject and the route of administration. In some embodiments, the dosage of the compound will vary from about 1 μg to about 10 mg per kilogram of body weight of the subject. In other embodiments, the dosage will vary from about 3 μg to about 1 mg per kilogram of body weight of the subject.

The compositions may be administered to a subject as frequently as several times daily, or it may be administered less frequently, such as once a day, twice a day, thrice a day, once a week, twice a week, thrice a week, once every two weeks, twice every two weeks, thrice every two weeks, once a month, twice a month, thrice a month, or even less frequently, such as once every several months or even once or a few times a year or less. The frequency of the dose will be readily apparent to the skilled artisan and will depend upon any number of factors, such as, but not limited to, the type and severity of the disease being treated, the type and age of the subject, etc. The formulations of the pharmaceutical compositions may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single-or multi-dose unit.

Individuals to which administration of the pharmaceutical compositions of the invention is contemplated include, but are not limited to, humans and other primates, mammals including commercially relevant mammals such as non-human primates, cattle, pigs, horses, sheep, cats, and dogs.

These compositions described herein are by no means all inclusive, and further modifications to suit the specific application will be apparent to the ordinary skilled artisan. Moreover, the effective amount of the compositions can be further approximated through analogy to compounds known to exert the desired effect.

The present invention is further illustrated in the following Examples. It should be understood that these Examples, while indicating exemplary embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Reinvigorating Effector Functions of Cytotoxic Immune Cells to Combat HIV-1 Using CRISPR-Cas9 Platform

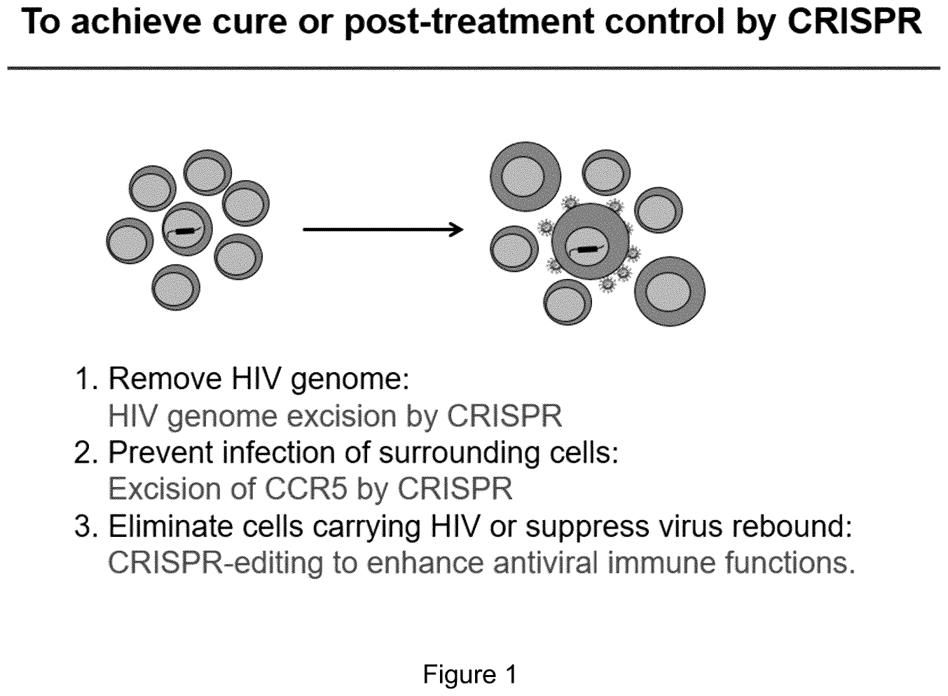

Achieving cure or post-treatment control by CRISPR comprises (1) removing HIV genome (HIV genome excision by CRISPR), (2) preventing infection of surrounding cells (excision of CCR5 by CRISPR), (3) eliminating cells carrying HIV or suppressing virus rebound (CRISPR-editing to enhance antiviral immune functions) (FIG. 1).

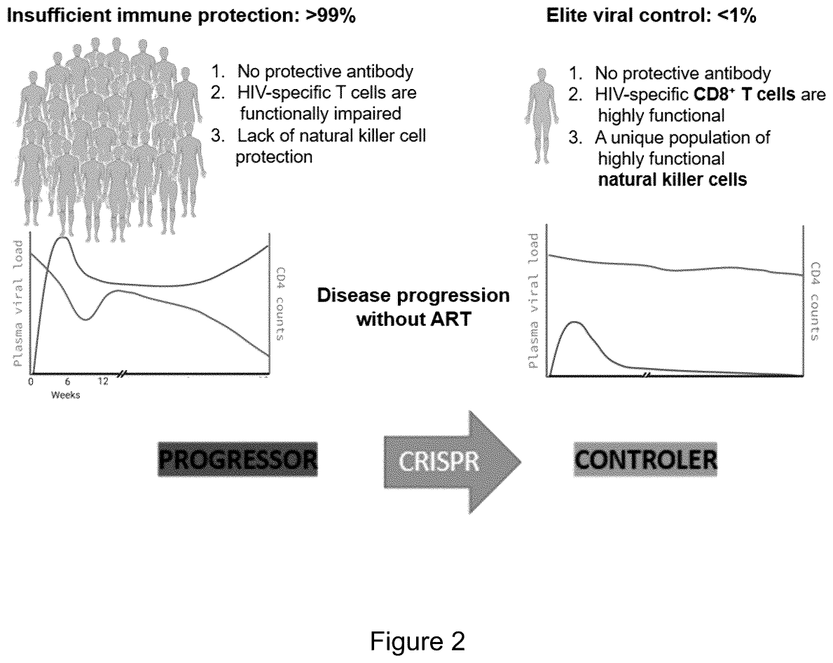

Insufficient immune protection (>99% of the population) is characterized by: no protective antibody, HIV-specific T cells are functionally impaired, lack of natural killer cell protection (FIG. 2). Elite viral control (<1% of the population) is characterized by: no protective antibody, HIV-specific CD8+ T cells are highly functional, a unique population of highly functional natural killer cells (FIG. 2).

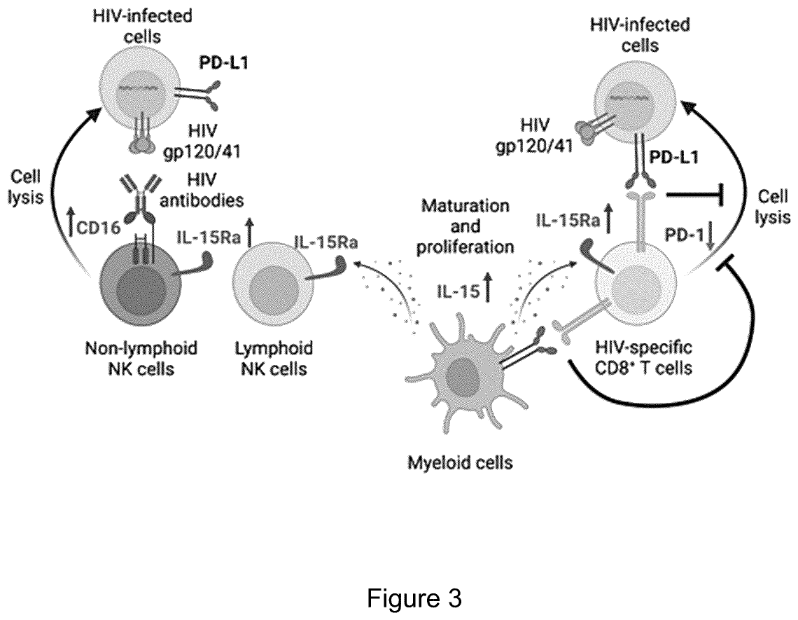

Gene(s) to be down-regulated by CRISPR comprise PD-1 (“taking breaks off”); gene(s) to be up-regulated by CRISPR comprise IL-15Ra, IL15, and CD16a (“pressing the gas”) (FIG. 3).

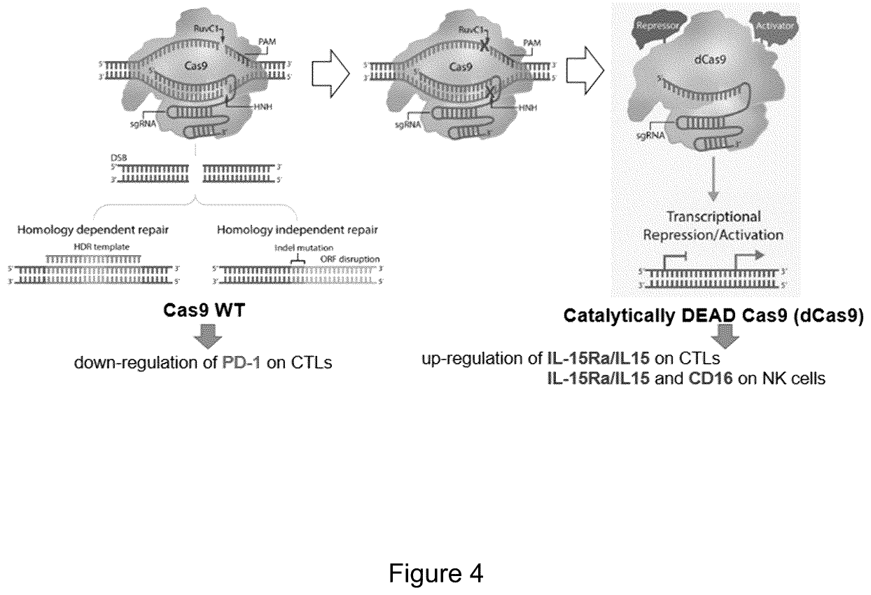

Repurposing CRISPR as an RNA-guided platform for sequence-specific control of gene expression comprises down-regulation of PD-1 on CTLs using WT Cas9, upregulation of IL-15Ra/IL15 on CTLs using catalytically dead Cas9 (dCas9), and upregulation of IL-15Ra/IL15 and CD16 on NK cells using dCas9 (FIG. 4).

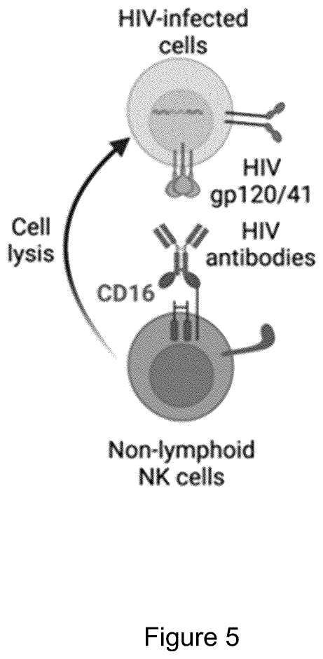

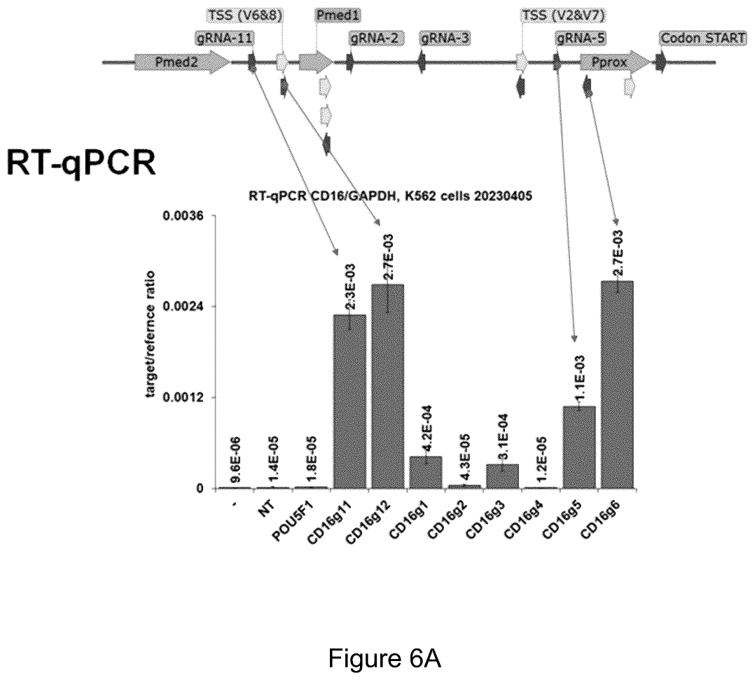

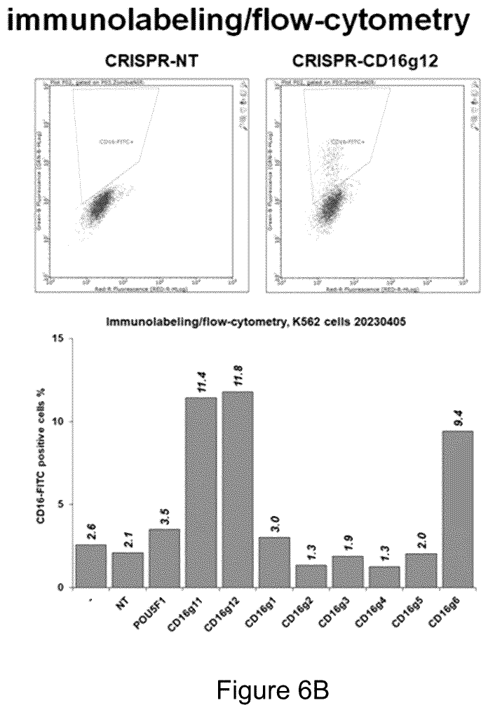

As depicted in FIG. 5, rationale for the reversal of NK exhaustion and increase NK killing by modulation of CD16 by CRISPR: NK cells play key role in clearance of viral infections via antibody-dependent cellular toxicity (ADCC), NK cells recognize antibody covered infected cells using CD16, binding activates cytokine release, degranulation and cytotoxicity, and immature NK cells in lymph nodes lack CD16 expression. CRISPR technology can be used to augment NK cells, by inducing/upregulating the CD16 receptor expression to maximize their efficiency. gRNA screen in K562-dCas9-VPR erythromyeloid cell line (CD16a) (FIG. 6A); qRT-PCR and immunolabeling/flow cytometry (FIG. 6B).

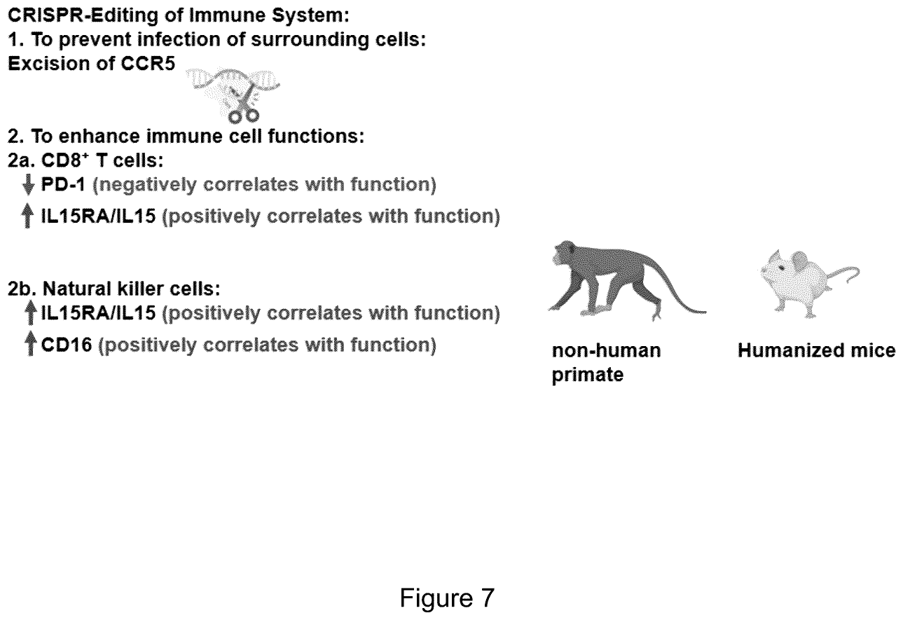

FIG. 7 presents molecular targets for immune editing by CRISPR, for example, in humanized mice, non-human primates, etc. CRISPR-editing of immune system to prevent infection of surrounding cells via excision of CCR5. To enhance immune cell functions of CD8+T cells: decrease in PD-1 (negatively correlates with function), increase in IL15RA/IL15 (positively correlates with function); to enhance immune cell functions of Natural killer cells: increase in IL15RA/IL15 (positively correlates with function); increase in CD16 (positively correlates with function).

### Example 2: CRISPR Modulation of CD16Ra Expression in Natural Killer Cells

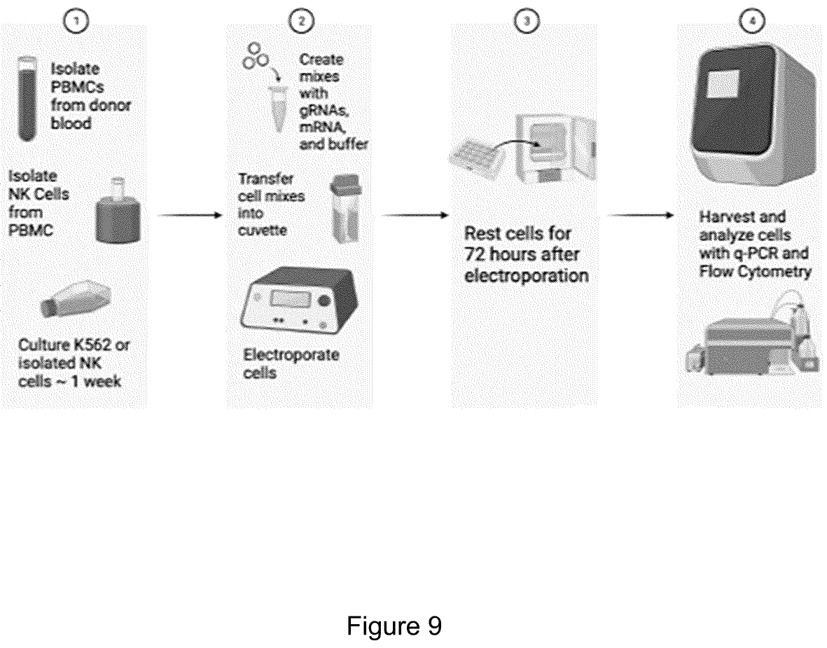

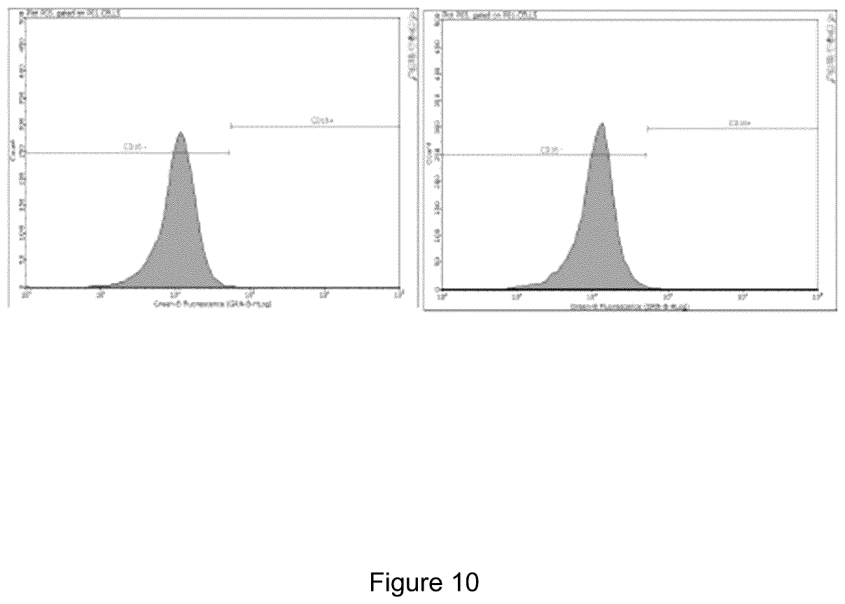

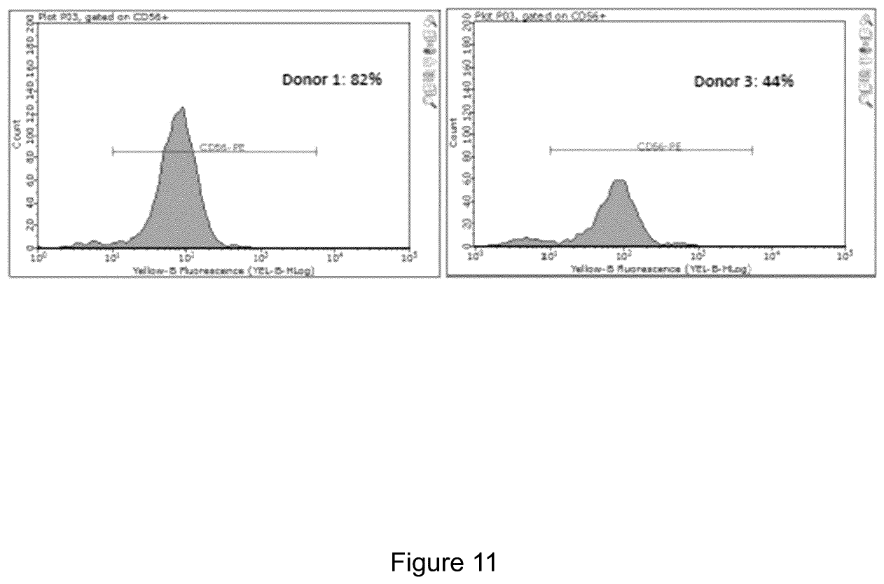

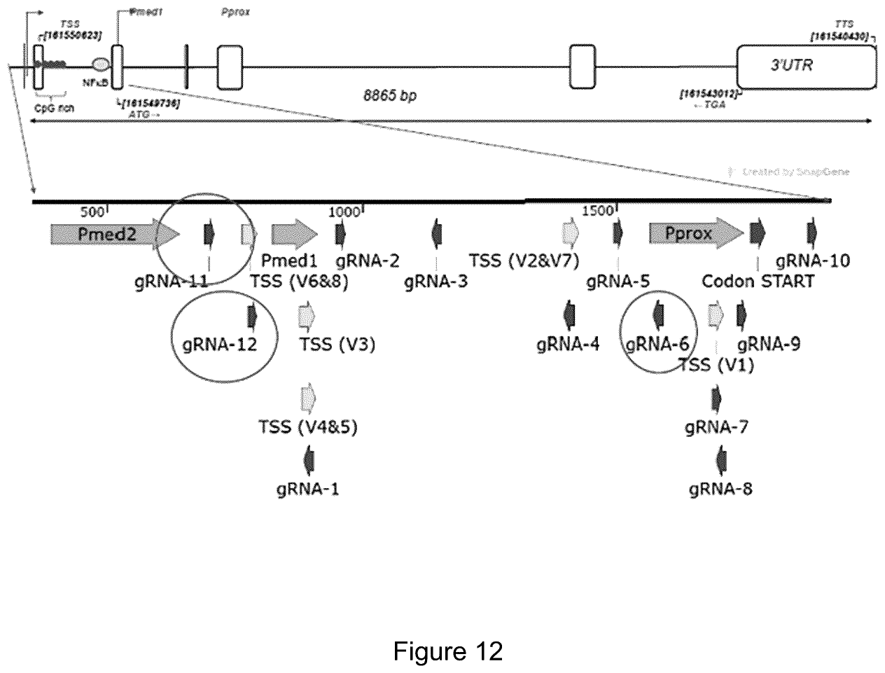

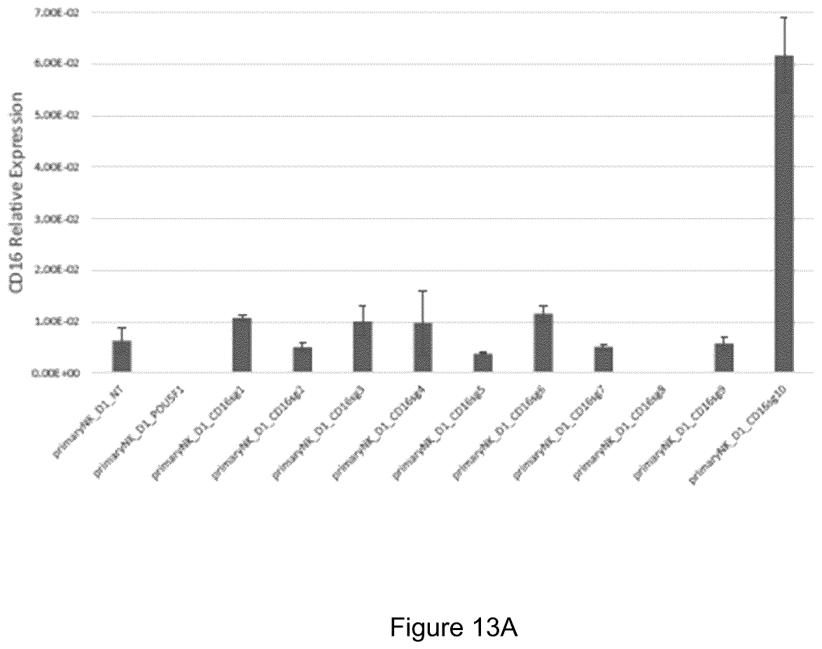

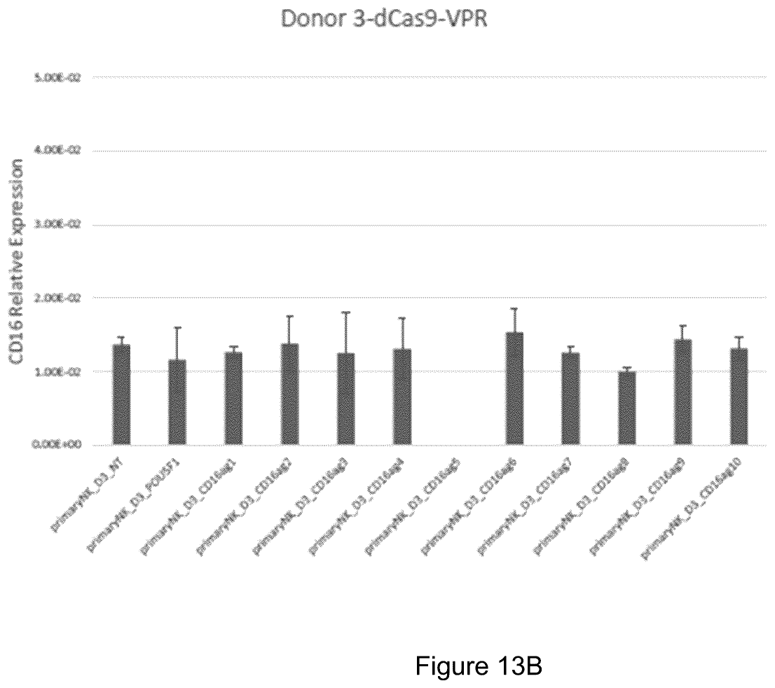

FIG. 6 and FIG. 8 through FIG. 13 depict the potential of using CRISPR-dCas9-VPR technology to upregulate the CD16 receptor on natural killer (NK) cells with the goal of increasing their efficiency and longevity. NK cells play a crucial role in antibody-dependent cellular cytotoxicity (ADCC), which involves the recognition and killing of virus-infected cells by antibodies bound to their surface. These antibodies can be recognized by the NK cell's CD16 receptor. In the context of HIV, ADCC has been shown to contribute to the control of viral replication and may also play a role in reducing the risk of HIV transmission in exposed uninfected individuals. It is determined that upregulating CD16 receptor expression will enhance NK cell-mediated cytotoxicity against target cells, leading to improved therapeutic outcomes in HIV and other diseases. Specific guide RNAs which bind to the promoter region of the FCGR3A gene coding for CD16 were screened on both K562 cells and human primary NK cells, and qPCR and flow cytometry were used to quantify the expression. Guides 6, 11, and 12 (SEQ ID NO: 6, SEQ ID NO: 11, and SEQ ID NO: 12, respectively) recruiting dCas9-VPR upstream of alternative transcription start sites showed the most promising upregulation of CD16 expression on both cell types. These results are encouraging; further optimization determines the optimal guide RNA(s) and assessment of the functional effects of CD16 upregulation on NK cell-mediated cytotoxicity. Nonetheless, this study provides valuable insights into the potential of CRISPR-dCas9-VPR technology for enhancing the therapeutic efficacy of NK cells, which may have important implications for the treatment of HIV and other diseases.

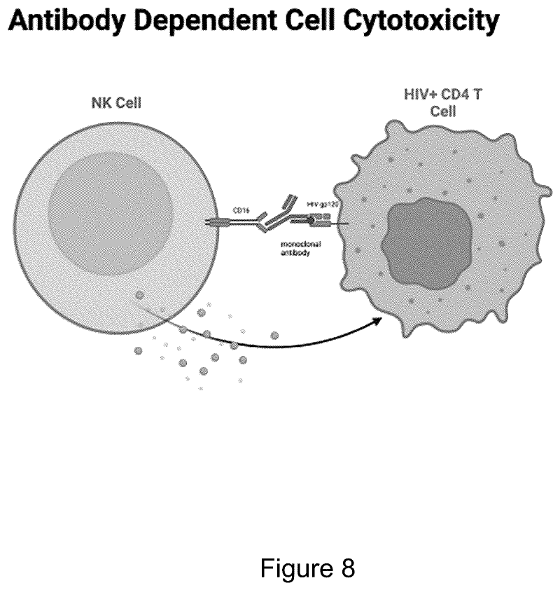

FIG. 8 depicts the procedure for Identification of most potent gRNA transactivation of FCGR3A gene expression in NK cells: check NK marker expression via flow cytometry, electroporate cells with dCas9-VPR mRNA+gRNAs and check CD16 expression via RT-qPCR and flow cytometry to determine best gRNA, validate best gRNA from above in primary NK cells, isolate NK cells from human blood donors, monitor lineage markers throughout expansion, electroporate cells with dCas9-VPR mRNA+gRNAs and check CD16 expression via RT-qPCR and flow cytometry to determine best gRNA.

Guides 6, 11, and 12 (SEQ ID NO: 6, SEQ ID NO: 11, and SEQ ID NO: 12, respectively) are optimal. These guides are located close to the transcription start site for FCG3Ra variants 1, 3, 6 and 8. Other variants can also be targeted to increase the killing efficiency of the NK cells. Promising guides are re-tested in K562 and NK92 MI cells and human primary cells. Killing efficiency of the cells is determined using an ADCC assay and an in vivo mouse model.

### Example 3: CRISPR-dCas9-Mediated Induction of CD16/FCGR3A Gene Expression in Human Natural Killer Cells

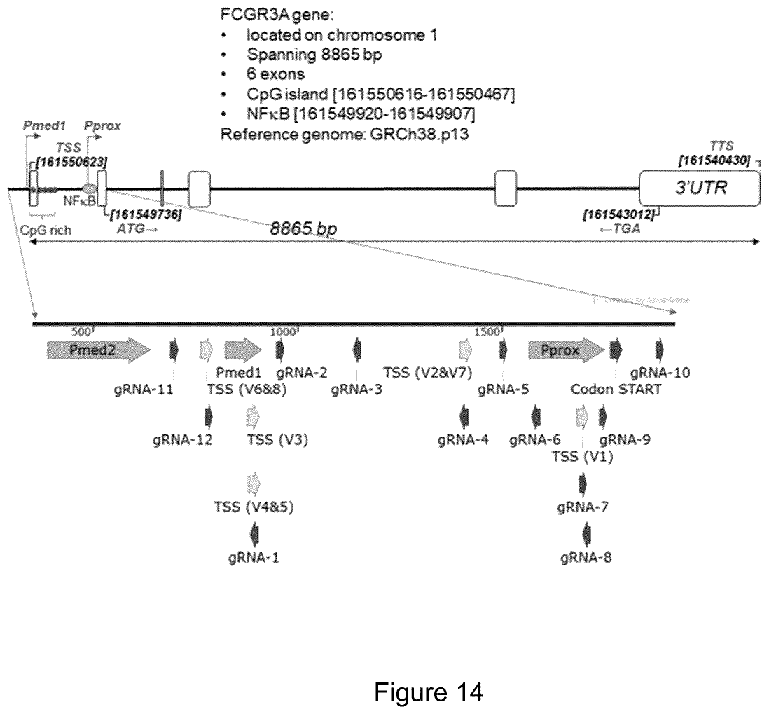

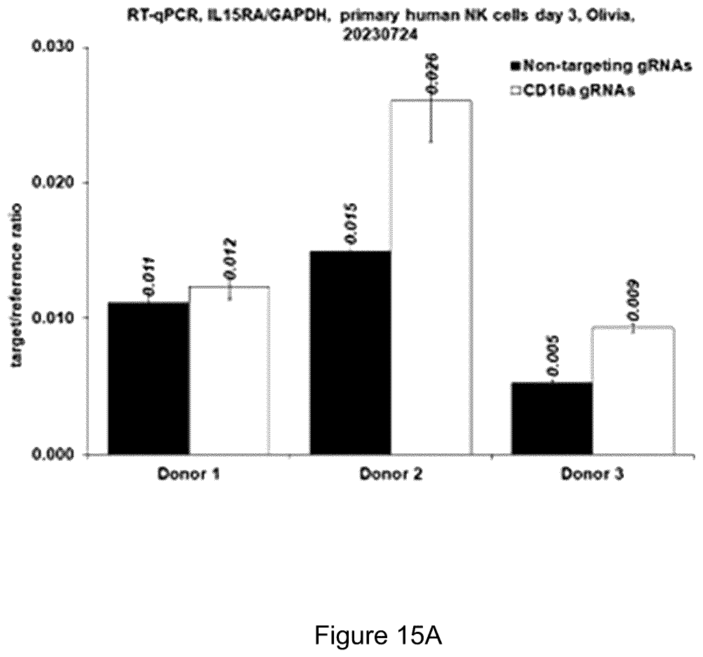

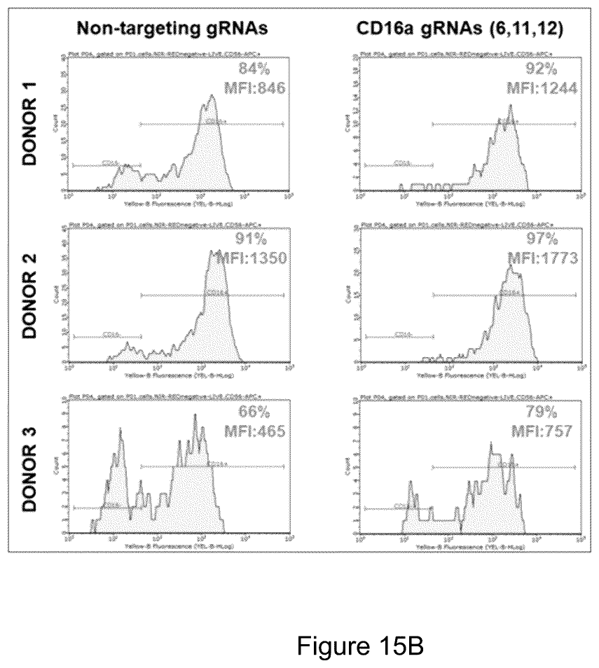

As depicted in FIG. 5, FIG. 6, FIG. 14, and FIG. 15, the potential of using CRISPR-dCas9-VPR technology to upregulate the CD16 receptor on natural killer (NK) cells to increase their efficiency and longevity was determined. NK cells play a crucial role in antibody-dependent cellular cytotoxicity (ADCC), which involves the recognition and killing of virus-infected cells by antibodies bound to their surface. These antibodies can be recognized by the NK cell's CD16 receptor. In the context of HIV, ADCC has been shown to contribute to the control of viral replication and may also play a role in reducing the risk of HIV transmission in exposed uninfected individuals.

Specific guide RNAs that bind to the promoter region of the FCGR3A gene coding for CD16 were screened on both K562 cells and human primary NK cells, and qPCR and flow cytometry were used to quantify the expression. Guides 6, 11, and 12 (SEQ ID NO: 6, SEQ ID NO: 11, and SEQ ID NO: 12, respectively) recruiting dCas9-VPR upstream of alternative transcription start sites showed the most promising upregulation of CD16 expression on both cell types. This study provides valuable insights into the potential of CRISPR-dCas9-VPR technology for enhancing the therapeutic efficacy of NK cells, which may have important implications for the treatment of HIV and other diseases. A set of three gRNAs potently inducing CD16 alpha expression in human NK cells were identified. CD16 induction/upregulation enhances the antibody-dependent cellular cytotoxicity (ADCC) ability of NK cells and has broad application in therapies based on NK cells.

Fc-dependent antibody effector functions are essential to the antibody-mediated elimination of residual HIV-infected cells. In the past 10 years, a variety of broadly reactive neutralizing antibodies (bNAb) have been isolated from a small subset of PWH and shown to act against a wide spectrum of viruses by targeting conserved regions on the HIV envelope trimer. Passive administration of bNAbs reduced plasma HIV-1 RNA in untreated viremic patients, and delayed virus rebound in ART-treated patients after treatment interruption. In the RV144 HIV clinical vaccine trial using ALVAC-HIV and AIDSVAX gp120 B/E, nNAbs targeting HIV-1 envelope gp120 V1V2 regions were associated with an estimated 31% protection against HIV acquisition, in the absence of neutralizing antibody responses and T cell responses. In addition to Fab regions which determine antibody neutralizing activity, antibody Fc regions are indispensable to antibody effector functions, which trigger the killing of antibody-coated cells. To kill HIV-infected cells, the antibody Fab region binds to the envelope protein on the surface of infected cells, while its Fc region engages with Fc-gamma receptors (FcγR) on the innate immune effector cells. In nonhuman primate and humanized mouse models, Fc effector functions are required for bNAb mediated clearance of infected cells, although it is not clear whether neutralizing activity also requires Fc functions. Fc-FcγR interaction can trigger the killing of infected cells through antibody-dependent cell-mediated cytotoxicity (ADCC) by natural killer (NK) cells (FIG. 5) and antibody-dependent cellular phagocytosis (ADCP) by phagocytes such as monocytes and macrophages. NK cells provide a rapid response to HIV infection and contribute to antibody mediated protection, but NK cell effector functions are impaired in active and chronic HIV infection, and only partially restored by ART. NK cells only express one FcγR, CD16. The lymphoid tissue resident NK cells are predominantly CD56+CD16−, and thus do not have ADCC function. Since lymphoid tissues are the most important anatomical reservoirs for latent HIV-1, a CRISPR-based approach is used to induce CD16 expression in NK cells.

Design and Selection of CRISPR gRNAs

The human FCGR3A, also known as CD16, CD16-II, CD16A, FCG3, FCGR3, FCGRIII, FCR-10, FCRIII, FCRIIIA, FcGRIIIA, IGFR3, IMD20 (chromosome 1) gene coding sequence including promoter region upstream of transcription start sites were screened using the CRISPR design tools (IDT & CRISPOR) for the presence of gRNAs binding sites for SpCas9 (NGG PAM sequence). A set of 12 candidate gRNAs targeting promoter of the human FCG3RA gene (FIG. 14 and Table 1) and having the highest ON-target efficiency and the lowest OFF-target scores were selected. Testing of CRISPR gRNAs targeting CD16/FCGR3A in K562-dCas9-VPR cell line. K562 lymphoblast cell line stably expressing dCas9-VPR (Horizon) was electroporated with synthetic gRNAs, including non-targeting gRNA as a control. After three days, the mRNA and surface protein expression of CD16 was examined by RT-qPCRs (FIG. 6A) and immunolabeling/flow cytometry (FIG. 6B), respectively. Experiments were repeated one more time. Based on the results lead gRNAs showing the highest and repeatable induction of the target gene expression were identified: g6, g11, and g12 (SEQ ID NO: 6, SEQ ID NO: 11, SEQ ID NO: 12, respectively) for CD16 (FIG. 6A and FIG. 6B and Table 1). Validation of lead CRISPR gRNAs in primary human NK cells. The mix of lead gRNAs was co-transfected with mRNA for dCas9-VPR (Horizon) into primary human NK cells isolated from the blood of three healthy donors (deidentified). As shown in FIG. 15 lead CD16 gRNA mix resulted in induction of CD16 expression in NK cells on both RNA (FIG. 15A) and protein level (FIG. 15B).

Functional assays were performed to show that dCas9-VPR-mediated induction of CD16 in primary human NK cells leads to increased proliferation and release of immunomodulating cytokines such as interferon-gamma (INFγ) and increased cytotoxic killing of the target HIV-1 infected cells. This approach was tested in vivo in a humanized mice model called MISTRG. Finally, the experiments using HIV-1-infected, ART-treated MISTRG mice were performed. The transience of the induction lowers the risk of aberrant activation of the immune system. The delivery of dCas9-VPR/gRNAs specifically into NK cells is ideal.

This is the first-ever study examining using the CRISPR-dCas9 platform to induce/upregulate expression on CD16 in NK cells. Recently reported upregulation of CD16 in NK cells for cancer therapy was achieved by knock-in of CD16 (Clara et al., 2022, J Immunother Cancer, 10: e003804). The advantage of this approach is that it uses a natural way of inducing CD16 expression in NK cells by targeting endogenous promoter of FCGR3A gene which should result in the transient production of physiological levels of CD16. dCas9-VPR/gRNAs delivery can be customized to specifically target NK cells reducing the risk of adverse effects related to systemic administration and global activation of the immune system.

Treatment of HIV-1 disease (to eliminate residual HIV-1-infected cells). NK cells provide a rapid response to HIV infection and contribute to antibody-mediated protection, but NK cell effector functions are impaired in active and chronic HIV infection, and only partially restored by ART. NK cells only express one FcγR, CD16. The lymphoid tissue resident NK cells are predominantly CD56+CD16−, and thus do not have ADCC function. CRISPR-based induction/upregulation of CD16 expression in NK cells will enable them and enhance the ADCC ability required for bNAb-mediated clearance of infected cells. CD16-dependent NK function augments the beneficial in-vivo activity of neutralizing antibodies (Mikulak et al., 2017, AIDS, 17: 2317-2330; Forthal et al., 2018, AIDS, 17: 2439-2451; Duan et al., 2022, Front Immunol., 13: 842746). Other chronic infection conditions associated with NK cell dysfunction or exhaustion. The same approach, CRISPR-mediated upregulation of CD16 expression in NK cells can be applied to other than HIV-1 chronic viral infections such as HBV, HCV, HTLV-1, and influenza and non-viral chronic infections such as tuberculosis, Listeria, malaria, toxoplasmosis, and leishmaniasis. Also, as mentioned above it can enhance vaccine-elicited immunity by augmenting the number, ADCC function, maintenance, and longevity of NK cells. 3. Cancer immunotherapy. Targeted cancer immunotherapies (such as bi-, tri-, and multispecific antibodies) redirecting immune effector cells to eliminate malignantly transformed cells require functional NK cells able to optimally perform their ADCC function (Pinto et al., 2022, Trends Immunol., 11: 932-946; Myers et al., 2021, Nat Rev Clin Oncol., 2: 85-100; Capuano et al., 2021, Cancers (Basel); 10: 2500). The same approach can be used to boost the functions of CAR-NK cells.

### Example 4: CRISPR-dCAS9-Mediated Induction of CD16/FCGR3A Gene Expression in Human Natural Killer Cells

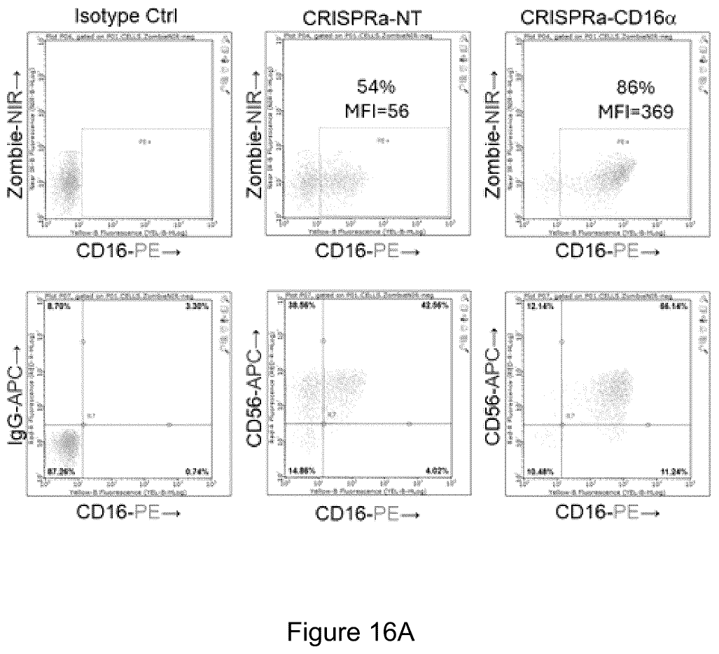

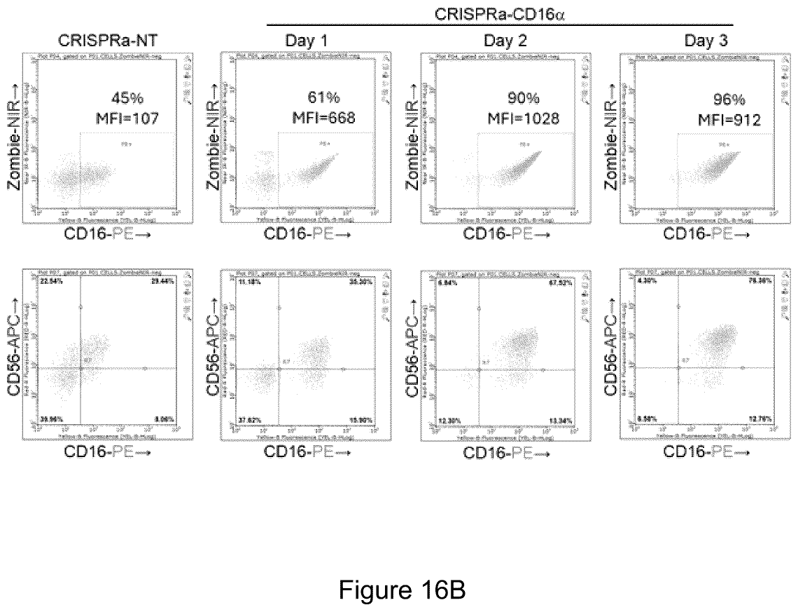

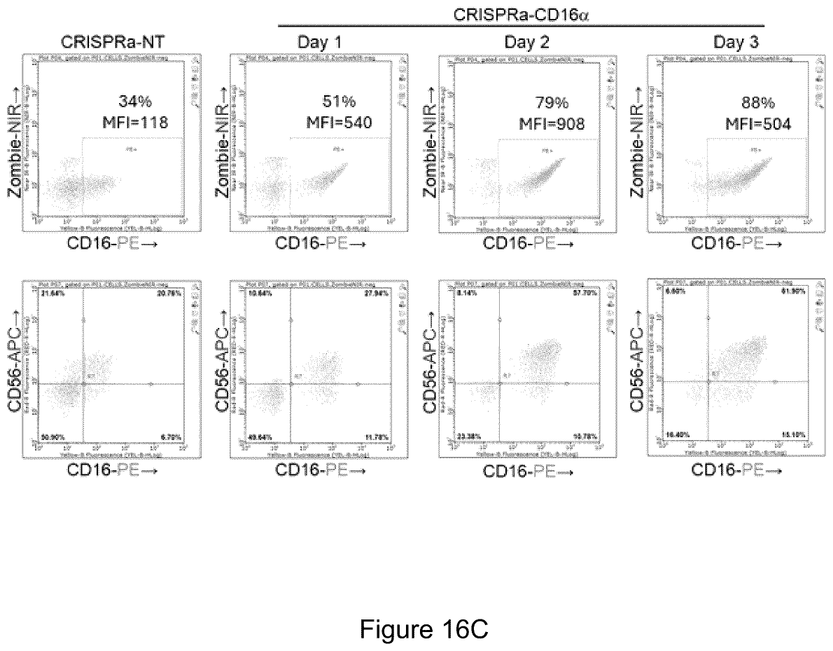

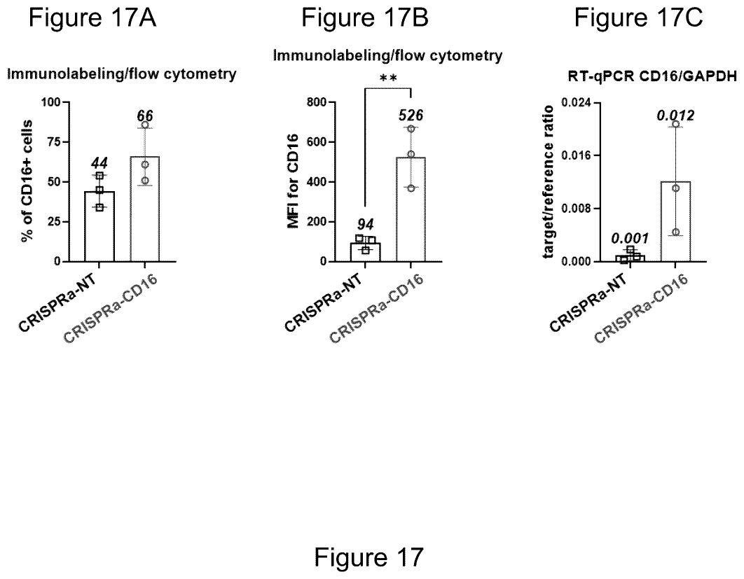

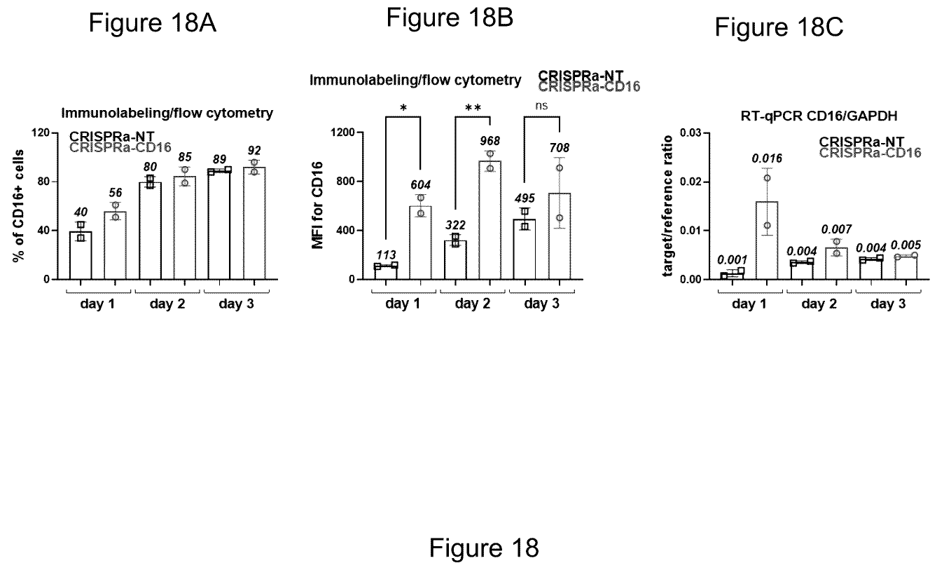

As depicted in FIG. 16 through FIG. 18, dCas9-VPR mRNA/gRNA electroporation leads to robust induction of CD16 mRNA and protein expression in primary human NK cells from different donors. The effects are highest at day 1 but are still detectable after 3 days in culture.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A genome editing system comprising a targeted transcriptional activator comprising a catalytically dead CRISPR Cas protein linked to a transcription activation domain, and at least one sgRNA molecule comprising nucleotide sequence that is complementary to the promoter of CD16.

2. The genome editing system of claim 1, wherein the at least one sgRNA molecule comprising nucleotide sequence that is complementary to the promoter of CD16 is selected from the group consisting of SEQ ID NO: 1-12.

3. The genome editing system of claim 2, wherein the at least one sgRNA molecule comprising nucleotide sequence that is complementary to the promoter of CD16 is selected from the group consisting of SEQ ID NO: 6, SEQ ID NO: 11, and SEQ ID NO: 12.

4. The genome editing system of claim 1, wherein the system comprises an mRNA molecule encoding the catalytically dead CRISPR Cas protein linked to a transcription activation domain.

5. The genome editing system of claim 1, wherein the transcription activation domain comprises a fusion protein comprising VP64, p65 and Rta.

6. A method of increasing the expression of CD16 in a subject, comprising administering to the subject:
a) a genome editing system comprising a targeted transcriptional activator comprising a catalytically dead CRISPR Cas protein linked to a transcription activation domain; and
b) at least one sgRNA molecule comprising nucleotide sequence that is complementary to the promoter of CD16.

7. The method of claim 6, wherein the at least one sgRNA molecule comprising nucleotide sequence that is complementary to the promoter of CD16 is selected from the group consisting of SEQ ID NO: 1-12.

8. The method of claim 6, wherein the system comprises an mRNA molecule encoding the catalytically dead CRISPR Cas protein linked to a transcription activation domain.

9. The method of claim 6, wherein the transcription activation domain comprises a fusion protein comprising VP64, p65 and Rta.

10. A method of increasing the proliferation of immune cells in a subject, comprising administering to the subject:
a) a genome editing system comprising a targeted transcriptional activator comprising a catalytically dead CRISPR Cas protein linked to a transcription activation domain; and
b) at least one sgRNA molecule comprising nucleotide sequence that is complementary to the promoter of CD16.

11. The method of claim 10, wherein the at least one sgRNA molecule comprising nucleotide sequence that is complementary to the promoter of CD16 is selected from the group consisting of SEQ ID NO: 1-12.

12. The method of claim 10, wherein the system comprises an mRNA molecule encoding the catalytically dead CRISPR Cas protein linked to a transcription activation domain.

13. The method of claim 10, wherein the transcription activation domain comprises a fusion protein comprising VP64, p65 and Rta.

14. The method of claim 10, wherein the immune cell is selected from the group consisting of a T cell, natural killer (NK) cell, myeloid cell, antigen presenting cell, dendritic cell, macrophage, and B cell.

15. The method of claim 10, wherein the immune cell is a NK cell.

16. A method of treating or preventing a disease or disorder in a subject, comprising administering to the subject:
a) a genome editing system comprising a targeted transcriptional activator comprising a catalytically dead CRISPR Cas protein linked to a transcription activation domain; and
b) at least one sgRNA molecule comprising nucleotide sequence that is complementary to the promoter of CD16.

17. The method of claim 16, wherein the at least one sgRNA molecule comprising nucleotide sequence that is complementary to the promoter of CD16 is selected from the group consisting of SEQ ID NO: 1-12.

18. The method of claim 16, wherein the system comprises an mRNA molecule encoding the catalytically dead CRISPR Cas protein linked to a transcription activation domain.

19. The method of claim 16, wherein the transcription activation domain comprises a fusion protein comprising VP64, p65 and Rta.

20. The method of claim 16, wherein the disease or disorder is cancer or an infectious disease.

21. The method of claim 16, wherein the disease or disorder is HIV infection or AIDS.


SEQUENCE LISTING (12 sequences)

SEQ ID NO: 1 - DNA, 23 bp, synthetic construct
  source 1..23: mol_type = other DNA
gtctgtcagcctaggagccgggg

SEQ ID NO: 2 - DNA, 23 bp, synthetic construct
  source 1..23: mol_type = other DNA
ggacctgggtaacacgaggaagg

SEQ ID NO: 3 - DNA, 23 bp, synthetic construct
  source 1..23: mol_type = other DNA
tcctgggatatgtaatccacagg

SEQ ID NO: 4 - DNA, 23 bp, synthetic construct
  source 1..23: mol_type = other DNA
atccacccagcaccaagaatggg

SEQ ID NO: 5 - DNA, 23 bp, synthetic construct
  source 1..23: mol_type = other DNA
gctcagccacagacctttgaggg

SEQ ID NO: 6 - DNA, 23 bp, synthetic construct
  source 1..23: mol_type = other DNA
agggagccccaccatagaacagg

SEQ ID NO: 7 - DNA, 23 bp, synthetic construct
  source 1..23: mol_type = other DNA
ttcctcctgtctagtcggtttgg

SEQ ID NO: 8 - DNA, 23 bp, synthetic construct
  source 1..23: mol_type = other DNA
agggaccaaaccgactagacagg

SEQ ID NO: 9 - DNA, 23 bp, synthetic construct
  source 1..23: mol_type = other DNA
gtgacttgtccactccagtgtgg

SEQ ID NO: 10 - DNA, 23 bp, synthetic construct
  source 1..23: mol_type = other DNA
tacgaatgagactcccacgaagg

SEQ ID NO: 11 - DNA, 23 bp, synthetic construct
  source 1..23: mol_type = other DNA
agatgtccacgaatccactgggg

SEQ ID NO: 12 - DNA, 23 bp, synthetic construct
  source 1..23: mol_type = other DNA
taaccaacgtggggttacggggg